(19) United States
(12) Patent Application Publication
(10) Pub. No.: US 2026/0348969 A1
(43) Pub. Date: Nov. 5, 2026

(54) SCALABLE PLATFORM FOR SINGLE CELL PROTEOMICS VIA DIGITIZED DETECTION OF PROTEINS AND THEIR PROTEOFORMS

(71) Applicant: Northwestern University, Evanston, IL (US)

(72) Inventors: Neil L. Kelleher, Evanston, IL (US); Pei Su, Evanston, IL (US); Michael AR Hollas, Evanston, IL (US); Jared Otto Kafader, Evanston, IL (US)

(21) Appl. No.: 19/487,993

(22) PCT Filed: May 31, 2024
(86) PCT No.: PCT/US2024/031987

§ 371 Date: Nov. 25, 2025

Related U.S. Application Data
(60) Provisional application No. 63/505,404, filed on May 31, 2023.

(51) Int. Cl. G01N 33/68; G16B 40/10

(52) U.S. Cl. CPC G01N 33/6842; G01N 33/6848; G16B 40/10; G01N 2570/00

(57) ABSTRACT

Disclosed herein are systems and methods for single-cell proteoform imaging mass spectrometry. The method includes moving a liquid droplet comprising a solvent across a substrate having disaggregated cells thereon, sampling the moving liquid droplet with an ionization source, wherein sampling the moving liquid droplet comprises detecting a discrete cell extraction profile comprising a multiplicity of proteoform ions when the moving liquid droplet contacts a disaggregated cell on the substrate, aggregating a mass-domain spectrum from a plurality of discreate cell extraction profiles; assigning a single cell proteoform from the aggregated mass-domain spectrum; and identifying one or more proteoforms from the assigned the single cell proteoform.

### CROSS REFERENCE TO RELATED APPLICATIONS

This application claims benefit of priority to U.S. Application Ser. No. 63/505,404, filed May 31, 2023, the contents of which is incorporated in its entirety.

### STATEMENT REGARDING FEDERALLY SPONSORED RESEARCH

This invention was made with government support under CA246635 and GM108569 awarded by National Institutes of Health. The government has certain rights in the invention.

### BACKGROUND OF THE INVENTION

Single-cell proteome analysis (SCPA) seeks to capture the heterogeneity in cellular type, state and function obscured by population-averaged measurements of proteins. Proteins in single cells and subcellular structures can been mapped using different molecular probe-based methods including antibody-based optical microscopy at high spatial resolution and sensitivity. However, such approaches require prior knowledge of target proteins and lack the molecular specificity to target proteoforms,¹ limiting our ability to discover the exact forms of proteins present in different cell types. Therefore, a more precise characterization of normal and pathological states of cells comprising cellular neighborhoods in complex tissue currently evades us.²

Combining the specificity of proteoform analysis with single cell measurement presents major challenges. Unlike genomics platforms for single-cell RNA sequencing (scRNA-seq),³⁵ single-cell proteomics (SCP) relies upon an analyte detection paradigm without amplification to interrogate the complexity of the proteome.⁶ Since 2018, mass spectrometry (MS) has been advanced to perform SCPA, using ‘bottom-up’ platforms such as Single Cell ProtEomics by Mass Spectrometry (SCoPE-MS).⁷⁻¹¹ This work has increased the number of proteins that can identified and quantified using peptide-based workflows,¹² yet the number of cells processed in a given study is limited to a few hundred per study (data not shown).¹³ To increase throughput, avoid multi-step processing¹⁴ and protein digestion, a more direct sampling approach has the theoretical advantage of characterizing cells at the level of whole protocorms. However, a scheme for intact proteoform SCPA using ‘top-down’ MS¹⁵ sets requirements for sensitivity and protein identification that have yet to be met.

Therefore, there is a need for methods and systems for single cell proteomics with improved sensitivity and throughput.

### BRIEF SUMMARY OF THE INVENTION

Disclosed herein are systems and methods for identifying one or more proteoforms from a single cell. The method includes moving a liquid droplet including a solvent across a substrate having disaggregated cells thereon, sampling the moving liquid droplet with an ionization source, wherein sampling the moving liquid droplet comprises detecting a discrete cell extraction profile comprising a multiplicity of proteoform ions when the moving liquid droplet contacts a disaggregated cell on the substrate, aggregating a mass-domain spectrum from a plurality of discrete cell extraction profiles, assigning a single cell proteoform from the aggregated mass-domain spectrum, and identifying one or more proteoforms from the assigned the single cell proteoform.

Another aspect of the technology provides for systems for identifying one or more proteoforms from a single cell. The system includes a primary capillary tube configured to flow a solvent to a substrate having disaggregated cells thereon, a nanospray capillary tube configured for analyte transfer, wherein the primary capillary tube and the nanospray capillary tube are configured to form a liquid droplet bridging a junction between the primary capillary tube and the nanospray capillary tube, a movable stage, wherein the substrate having the disaggregated cells is disposed on the movable stage and the movable stage is configured to move the liquid droplet across the substrate, an ionization source configured to sample the liquid droplet, a detector configured to detect a discrete cell extraction profile comprising a multiplicity of proteoform ions, and one or more processors configured to aggregate a mass-domain spectrum from a plurality of discreate cell extraction profiles, assign a single cell proteoform from the aggregated mass-domain spectrum, identify one or more proteoforms from the assigned the single cell proteoform, and/or control the movable stage to move the liquid droplet across the substrate.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. The scPiMS workflow for proteoform detection and identification from single cells. Panel (a) shows the scanning approach (at left); snapshots at right show the relative location of a cell before (I), on (II), and after (III) analyte extraction by a liquid bridge (scale bar=50 μm). The extraction profile of the single cell (a “cellogram”) is shown at right. Mass spectrum of the ions collected from a single cell is shown in panel (b). The aggregated cellogram containing 586 single cell features in one run is shown in panel (c), and its corresponding aggregated mass spectrum is shown in panel (d). Panel (e) shows workflow for the main steps involved with the scPiMS process. Panel (f) shows ratio of single cell versus double and multiple cell features in the scPiMS run yielding the 10.8 k molecular features data set. Collector's curves for number of charge-assigned ions are show in panel (g), proteoforms detected algorithmically as well-formed isotopic distributions in panel (h), and proteoforms assigned to the rat hippocampal ion library using PAS in panel (i).

FIG. 2 shows mass domain spectra obtained from 10836 aggregated cells, single cells, and specific rat brain cell types. Panel (a) shows aggregated 10.8 k single cell spectrum in the 4-70 kDa mass range labeled with identified proteoforms. Insets show expanded regions around 21 kDa (orange box) and 14 kDa, with the latter capturing the proteoform landscape of Myelin basic proteins (blue box). Panels (b-e) show single-cell mass spectra (dark trace) showing the detection of single ions (top) of four selected scProteoforms and their theoretical isotopic distributions (bottom, mirrored in purple). Panels (f-i) show a set of 4 proteoform markers detected in scPiMS that differentiate and provide initial assignment of rat hippocampal cell types (508 neurons, 1538 astrocytes and 712 microglia). In each panel, the top spectrum shows an expanded spectral region of the ion library (light purple) and 10.8 k-cell aggregated dataset (black trace); the bottom three insets show proteoform spectral regions, all normalized to the same abundance scale, aggregated from 508 neurons, 1538 astrocytes and 712 microglia, respectively.

FIG. 3 shows a schematic of the experimental setup for scPiMSin targeted mode.

FIG. 4 shows example cellogram with 4 seconds in panel (a) and 8 seconds in panel (b) of proteoform signal at a flow rate of 500 and 180 nL/min, respectively.

FIG. 5 shows proteoform ion counts from 15 targeted single cells at MS injection times of 200 ms, 500 ms, and 800 ms.

FIG. 6 shows noise ion count per MS scan at two different HCD Pressure settings. The noise ions are obtained from a blank region of the high-throughput experiment where the probe scans across a region with no cells present; proteins could be present in blank regions of slides.

FIG. 7 shows comparison of thresholds for extraction of true single cellograms.

FIG. 8 shows targeted mode scPiMS. Panel (a) shows experimental setup containing cells on a glass slide, 900× optical microscope, and the nano-DESI probe configured with MS. Panel (b) shows a cartoon illustration of the targeted cell mode experiment before (top) and after (bottom) cell sampling is triggered. Panel (c) shows a snapshot of a single NIH 3T3 cell on the slide with the liquid bridge parked ~100 μm next to it prior to sampling. Panel (d) shows a cellogram of the targeted scPiMS experiment on the cell shown in panel (c). The data points highlighted in red and orange correspond to the scans within FWHM and scans with ion count more than 3 times of the baseline, respectively.

FIG. 9 shows optimization for scPiMS. Total proteoform ion counts determined for collections of ~10 single cells upon varying three different experimental variables: panel (a) shows nano-DESI extraction/ionization solvent containing 2% or 5% acetic acid; panel (b) shows different fixation protocols; panel (c) shows different MS injection times.

FIG. 10 shows scProteoforms and Proteoform Assignment Scores. Theoretical isotopic distribution (filled bars on the top), single cell spectrum (black trace in the middle), and scProteoform (dots with droplines on the bottom) of CH10 in panel (a), H2A2A in panel (b), and VIM in panel (c) for one targeted cell at 700 ms injection time and HCD gas pressure setting of 0.5. The number of single ions matched to each theoretical isotope, total number of ions in the scProteoform, and PAScores were labeled in the panels. On the bottom, each dot represents an individual ion.

FIG. 11 shows Proteoform Assignment Scores for scProteoforms as a function of ion injection time and HCD gas pressure. Summary of PAScore for three selected proteoforms, CH10, H2A2A, and VIM, at different MS injection times in panel (a) and HCD gas pressure settings in panel (b). Each dot represents the PAScore of the scProteoform for a targeted single cell.

FIG. 12 shows profiling ~500 single NIH 3T3 cells using scPiMS. Panel (a) shows total ion cellogram of the experiment. The dark bars on top indicate the positions where the 461 single cell cellograms were detected in the dataset. The PAScores of monoacetylated vimentin (VIM) of the 461 cells are shown at top. Panel (b) shows mass spectrum of NIH 3T3 cells from the aggregated 461-cell dataset. The insets show expanded regions of the spectrum corresponding to histone H2A & H2B and histone H1. The peaks with asterisks are phosphate adducts of the annotated proteoforms.

FIG. 13 shows estimating the throughput of scPiMS. Panel (a) shows statistical modeling of throughput of cells per day at different numbers of cells on a slide (dots) and number of possible single cell events (bars) using scPiMS probes of different sizes. The percentage labels indicate the calculated success rate for analysis of isolated, single cells. Panel (b) shows statistical modeling of total number of cells on slide (bars) and days to analyze these cells (purple triangles) using scPiMS probes of different sizes, if targeting ~90% of the cell population on a slide to be analyzed as single cells. Percentage success rates of single cell analysis are labeled in yellow text.

FIG. 14 shows ten targeted mode scPiMS experiments in panels (a-h, j-k) showing consistent single cell protein extraction kinetics. In each panel, the plot shows ion count chronogram. The medium-dark and light data points correspond to the scans with ion counts above FWHM and with ion counts >3 times that of the background scans, respectively.

FIG. 15 shows cellograms of single cells under different nano-DESI solvent conditions (0.6% (a), 2% (b), and 5% (c) acetic acid with 60%/40% acetonitrile/water). The baselines of the cellograms are offset for visualization. The different peak positions are due to the time lapse between sampling manipulation and data acquisition in targeted scPiMS experiments. 2% HAc gave the highest signal to noise ratio among these conditions.

FIG. 16 shows background individual ion counts collected by scanning on a blank area of the slide without cells with 2% (dark trace) and 5% (light trace) acetic acid in the nano-DESI solvent containing 60%/40% acetonitrile/water. Ion counts when encountering a single cell can be 10-20 times above noise levels.

FIG. 17 shows cellograms of NIH 3T3 cells prepared by different fixation protocols: panel (a) shows 100% cold ethanol into PBS buffer as a control; panel (b) shows 75%/25% cold ethanol/water into PBS buffer; panel (c) show removal of PBS incubation buffer prior to the addition of 100% cold ethanol. The blue arrow points to an observed cell that was potentially mechanically ruptured in the preparation process; panel (d) shows replacing PBS with ammonium acetate prior to 100% cold ethanol; panel (e) shows 95%/5% cold ethanol/glacial acetic acid into PBS buffer. The different peak positions are due to the time lapse between sampling manipulation and data acquisition in targeted cell scPiMS experiments.

FIG. 18 shows steps for different fixation protocols and photomicrograph of the fixed cells: panel (a) shows 100% cold ethanol into PBS buffer as a control; panel (b) shows 75%/25% cold ethanol/water into PBS buffer; panel (c) shows removal of PBS incubation buffer prior to the addition of 100% cold ethanol. The arrow indicates an observed cell that was potentially mechanically ruptured in the preparation process; panel (d) shows replacing PBS with ammonium acetate prior to 100% cold ethanol; panel (e) shows 95%/5% cold ethanol/glacial acetic acid into PBS buffer.

FIG. 19 shows background ion count on the glass slide at different injection times.

FIG. 20 shows total ion count of single cells at different HCD gas pressure settings.

FIG. 21 shows cellograms of single NIH 3T3 cells analyzed in raster scanning mode at different scan rates (8 μm/s, 10 μm/s, and 20 m/s). These cellograms were obtained from three different cells on the same slide in the same batch of preparation. In these conditions, the probe was in contact with the cells for 25 s, 20 s, and 10 s, respectively. These similar extraction profiles indicate that the extraction kinetics is not affected by the probe motion as long as a 10 s sampling time is reached. The post-cellogram baseline was higher than normal for these cells.

FIG. 22 shows stitched high-contrast optical image of the slide containing ~538 cells. The dots correspond to the spatial locations of the manually annotated cell features using QuPath software.

FIG. 23 shows cartoons of two adjacent grids that generates, in panel (a), two single cell events and, in panel (b), multiple cell event and a single cell event.

### DETAILED DESCRIPTION OF THE INVENTION

Disclosed herein are systems and methods for identifying one or more proteoforms from a single cell. Advantageously, these systems and methods enable high-sensitivity, high-resolution, and highly localized detection and identification of proteoforms of single cells over alternative approaches. The disclosed technology detects and identifies proteoforms from single cells by coupling a localized liquid-extraction surface sampling approach with individual ion mass spectrometry to achieve single-cell Proteoform imaging Mass Spectrometry (scPiMS).

The disclosed technology combines nanospray desorption electrospray ionization mass spectrometry imaging (nano-DESI MSI) with data acquisition and processing for individual ions for the proteoform-selective imaging of single cells spread across a substrate. Proteoforms are sampled as multiply charged ions distributed across multiple charge states. Instead of unresolved signals from overlapping charge states of protein mixtures typically observed for ensembles of ions, scPiMS provides information of individual protein ions in the mass domain even when their charge states overlap in the mass-to-charge domain. Enabled by direct charge assignment to individual ion signals, scPiMS generates mass-domain information of the individual proteoforms with resolution of peaks in each pixel of the imaging data. This allows for confident assignment of proteoform masses in each pixel of the imaging data.

The methods disclosed herein include moving a liquid droplet comprising a solvent across a substrate having disaggregated cells thereon; sampling the moving liquid droplet with an ionization source, wherein sampling the moving liquid droplet comprises detecting a discrete cell extraction profile comprising a multiplicity of proteoform ions when the moving liquid droplet contacts a disaggregated cell on the substrate; aggregating a mass-domain spectrum from a plurality of discreate cell extraction profiles, assigning a single cell proteoform from the aggregated mass-domain spectrum; and identifying one or more proteoforms from the assigned the single cell proteoform.

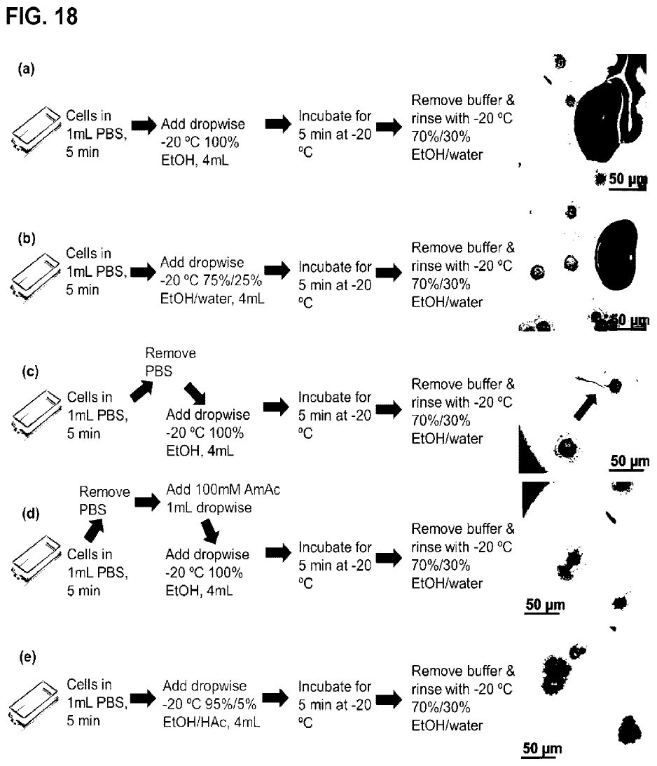

The disclosed technology may be applied to disaggregated cells. In some cases, disaggregated cells are spatially separated cells. As demonstrated in Example 1, disaggregated cells may be prepared from a tissue, organ, or organ system such as rat hippocampus. As demonstrated in Example 2, disaggregated cells may be prepared from cell culture such as NIH 3T3 mouse embryonic fibroblast cells. In some cases, the cells may be fixed. In some cases, the cells are fixed onto the substrate by suspending cells in a buffer, fixing the suspended cells onto the substrate with a fixation solution comprising ethanol, and washing the disaggregated cells fixed on the substrate with a wash solution comprising ethanol and water. In some cases, the cells may be fixed by suspending cells in a buffer, removing the buffer, fixing the suspended cells onto the substrate with a fixation solution including ethanol, and washing the disaggregated cells fixed on the substrate with a wash solution comprising ethanol and water. Several fixation methods are summarized in FIG. 18. In some cases, a cold 75%/25% ethanol/water mixture may be a preferred in situ fixation method.

In some cases, the disaggregated cells are adhered to a substrate. In some cases, the substrate may be further treated or functionalized to improve cell adhesion. For example, the substrate may be functionalized with poly-lysine-like moieties, functionalized with silane, or treated with an oxygen plasma. The substrate may include glass, indium tin oxide, or another substrate material suitable for supporting cells. In some cases, the substrate may be optically transparent to enable the visualization of disaggregated cells.

The disclosed technology includes moving a liquid droplet, also referred to as a ‘liquid bridge,’ comprising a solvent across a substrate. As described in the Examples below and as is well established in the art, nanospray desorption electrospray ionization (nano-DESI) utilizes a liquid bridge formed between two capillary tubes positioned near or on a substrate. A primary capillary supplies a liquid to maintain the dynamic liquid bridge between the junction of the two capillary tubes and the substrate surface. In some cases, the liquid bridge is maintained by flowing a liquid from the primary capillary tube. A second, nanospray capillary tube transfers at least a portion of the liquid droplet, and analytes therein, to an ionization source for subsequent mass spectrometry analysis. In some cases, a high voltage (e.g., several kV) is applied between the inlet of a mass spectrometer and the primary capillary, creating a self-aspirating nanospray. In some cases, the nanospray capillary tube transfers the liquid droplet, and analytes therein by instantaneous vacuum aspiration. As used herein, the term ‘nano-DESI probe’ refers to the primary capillary tube, the nanospray capillary tube and the liquid bridge between them.

The size of the liquid bridge (e.g., the volume of the liquid droplet, the surface area of the liquid droplet on the substrate, the diameter of the liquid droplet on the substrate) in nano-DESI can be adjusted according to the desired spatial resolution of the method. As described in the Examples, the liquid droplet may range from 25 to 400 μm in diameter, depending on the size of the capillaries used to fabricate the probe. In some cases, the liquid droplet may have a 200 μm diameter.

The primary capillary supplies a liquid to maintain the dynamic liquid bridge between the junction of the two capillary tubes and the substrate surface by supplying a liquid including a solvent at a flow rate. The flow rate may be between 50 and 1000 nL/min, between 75 and 750 nL/min, between 100 and 600 nL/min, between 140 and 500 nL/min, between 150 and 1000 nL/min, between 150 and 500 nL/min, or between 175 and 550 nL/min.

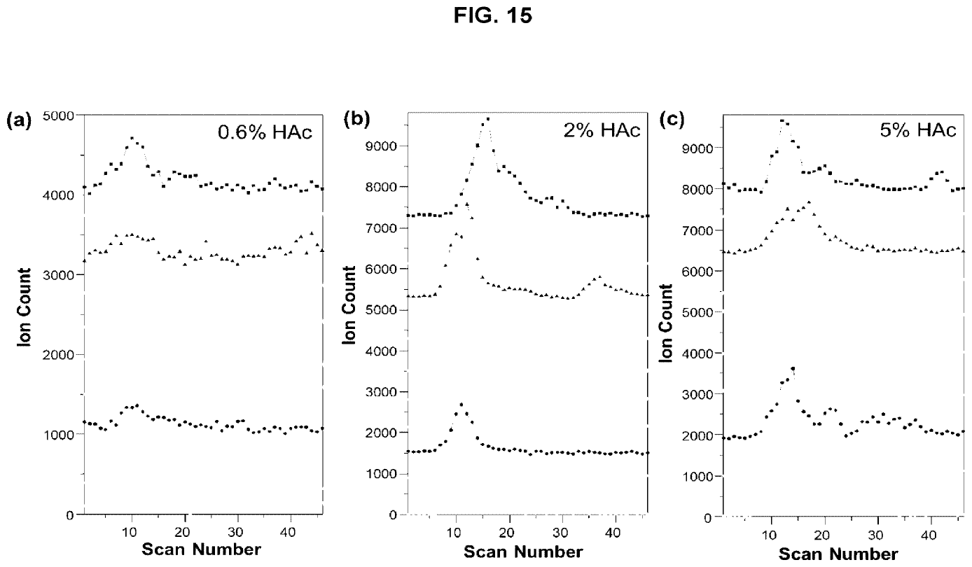

The composition of the liquid droplet may be selected to optimize the extraction and/or ionization of one or more analytes of interest from the liquid bridge, as shown in FIG. 15. In some cases, the composition of the liquid droplet may include a solvent to aid in extraction and/or ionization of proteins from the cell. Polar solvents, non-polar solvents, protic solvents, aprotic solvents, and combinations thereof may be used. For example, the liquid droplet may include acetonitrile, water, acetic acid, and combinations thereof. In some cases, the liquid droplet may include 40-80% acetonitrile, 20-60% water, and 0.1-10% acetic acid; 50-70% acetonitrile, 30-50% water, and 0.5-5% acetic acid; or 55-65% acetonitrile, 35-45% water, and 0.5-5% acetic acid. In some cases, the liquid droplet may contain 60%/40% acetonitrile/water prior to the addition of 2% acetic acid.

In another aspect, the technology disclosed herein includes moving the liquid droplet relative to a substrate having disaggregated cells thereon. Several approaches to moving the liquid droplet relative to the substrate can be contemplated, including keeping the droplet in a fixed location and mounting the substrate on a movable stage, thereby moving the substrate relative to the liquid droplet.

In some cases, the liquid droplet is moved across the substrate at a rate between 0.5 μm/s to 150 μm/s, between 1 μm/s to 100 μm/s, between 2 μm/s to 75 m/s, between 8 μm/s to 60 m/s, or between 20 μm/s to 50 μm/s. The rate of scanning the liquid droplet across the substrate may be optimized to maximize throughput and sufficient detection of one or more proteoform ions. For example, the liquid droplet can move across the substrate at a rate that when the liquid droplet leaves the disaggregated cell the detection of the one or more proteoform ions is waning. In some cases, the detection of the one or more proteoform ions is waning when the initial drastic rise in protein signal has begun to exponentially decay over time. In other cases, the detection of the one or more proteoform ions is waning when the initial drastic rise in protein signal has returned to baseline, indicating that the extractable proteins were captured. In some cases, the detection of the one or more proteoform ions is waning when the protein signal reaches 1% of the initial drastic rise in protein signal, 5% of the initial drastic rise in protein signal, 10% of the initial drastic rise in protein signal, 20% of the initial drastic rise in protein signal, 30% of the initial drastic rise in protein signal, or 40% of the initial drastic rise in protein signal.

As described in Examples below, the methods and systems disclosed herein may be operated at least two operational modes. In some instances, targeted mode may be used where a single cell of interest is identified in a first step. In a second step, the nano-DESI probe is brought in contact with the substrate so that the liquid bridge contacts at least a portion of the single cell of interest. Optionally, the nano-DESI probe may be brought in contact with the substrate near the cell (e.g., about 50 micrometers from the cell) initially and the movable stage is operated so that the liquid bridge comes into contact with at least a portion of the single cell of interest.

In another aspect, the duration of time at least a portion of the cell is in contact with the liquid bridge is the extraction time, also referred to herein as ‘exposure time.’ The extraction time may range from 0.25 seconds to 120 seconds. In some cases, the extraction time is about 5 to about 30 seconds.

Alternatively, in a scanning mode, the nano-DESI probe may be scanned across the substrate. In some cases scanning may include a pattern, such as in parallel lines at a constant linear velocity (i.e., raster scanning). Raster scanning rate is a function of nano-DESI probe size and exposure time, as described in detail in Examples 1-2 and Equations 1 and 9.

The methods and systems disclosed herein provide for ionization of at least a portion of the liquid droplet with an ionization source (e.g., sampling the moving liquid droplet with an ionization source). There are several suitable ionization sources already known in the art, including nano-DESI as described above, which performs both sampling and ionization. In some cases, sampling the moving liquid droplet includes detecting a discrete cell extraction profile including a multiplicity of proteoform ions when the moving liquid droplet contacts a disaggregated cell on the substrate.

In another aspect, also disclosed herein are a detector configured to detect a discrete cell extraction profile comprising a multiplicity of proteoform ions. Detection of a discrete cell extraction profile may be performed by mass spectrometry instrumentation and techniques already known in the art, including individual ion mass spectrometry (I²MS) mode on a mass spectrometer as described in the Examples below. As used herein, a discrete cell extraction profile is also referred to as a ‘cellogram,’ which is the measured burst of ions extracted from a single cell as the nano-DESI probe samples and ionizes the liquid droplet positioned over at least part of a single cell, plotted as an extraction curve.

In another aspect, operating parameters of individual ion mass spectrometry (I²MS) may be optimized for tuned such that ions in one detection period are collected in the individual ion regime (i.e., to maximize the population of individual ion fragments), which gives a singular ion signal at a defined m z (or frequency) value. In some cases, injection time of proteoform ions generated by nano-DESI and higher-energy collision dissociation gas pressure may be tuned. In some cases, the injection time is about 100 to 1000 ms, 200 to 900 ms, or 200 to 800 ms. The higher-energy collisional dissociation gas pressure may range from 0.1 to 1.0, 0.2 to 0.8, or 0.4 to 0.6.

The technology disclosed herein also provides for assigning single cell proteoforms, including aggregating a mass-domain spectrum from a plurality of discrete cell extraction profiles (i.e., cellograms) and identifying one or more proteoforms from the assigned the single cell proteoform. Each of these steps are described in more detail in Examples 1 and 2 below. The single cell proteoform is assigned by assigning a proteoform assignment score (PAS) based on mass accuracy and relative abundance of isotopologue masses of ions. The proteoform assignment score is

$$PAS = 1 - {\prod }_{k = 1}^{n} [ 1 - ( {P}_{isotopologue} \times {P}_{mass error} ) ]$$

where P_{isotopologue} is the expected relative intensity of the matching isotopologue and P_{mass error} is a probability score for the mass error-based between the observed individual ion mass and the theoretical mass of the isotopologue. The proteoform assignment score utilizes the cumulative distribution function (CDF) of a normal distribution with a mean and standard deviation determined by the theoretical mass and width of an isotopic peak, respectively. The P_{mass error} is

$${P}_{mass error} ∝ \{ \begin{matrix}CDF ( {m}_{ion} ) & {m}_{ion} < {m}_{iso} \\ [ 1 - CDF ( {m}_{ion} ) & {m}_{ion} > {m}_{iso}\end{matrix}$$

where mᵢₒₙ is the mass of the individual ion and mᵢₛₒ is the theoretical mass of the matched isotopologue. The one or more proteoforms are identified by an intact mass tag search against candidate proteoform and/or a deconvolution algorithm.

The systems and methods disclosed herein include one or more processors configured to aggregate a mass-domain spectrum from a plurality of discreate cell extraction profiles, assign a single cell proteoform from the aggregated mass-domain spectrum, identify one or more proteoforms from the assigned the single cell proteoform, and/or control the movable stage to move the liquid droplet across the substrate. In some cases, the one or more processors are configured to aggregate a mass-domain spectrum from a plurality of discreate cell extraction profiles, assign a single cell proteoform from the aggregated mass-domain spectrum, and identify one or more proteoforms from the assigned the single cell proteoform.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

Disclosed herein is single-cell Proteoform imaging Mass Spectrometry (scPiMS) to enable facile proteoform analysis from single cells spread onto substrates, such as glass slides.²⁶ Example 1 demonstrates scPiMS profiling of 10,836 cells disaggregated from rat hippocampus was performed in 10 days, yielding detection of 472 single-cell proteoforms with over 160 identified including well-known markers like ENOG (neurons) and GFAP isoforms (astrocytes) that striate brain cell types without antibody staining. Utilizing selected cell-type markers, scPiMS was able to stratify 2756 individual cells into three different cell types; astrocytes (1536), microglia (712), and neurons (508). scPiMS allows for advancement of single cell biology.

### The Single-Cell PiMS Workflow

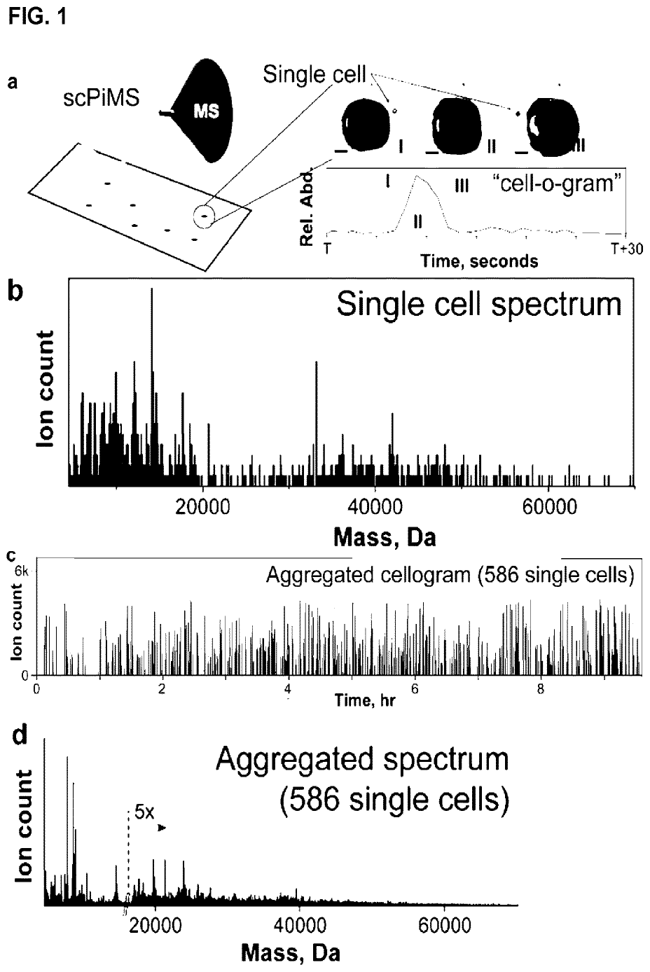

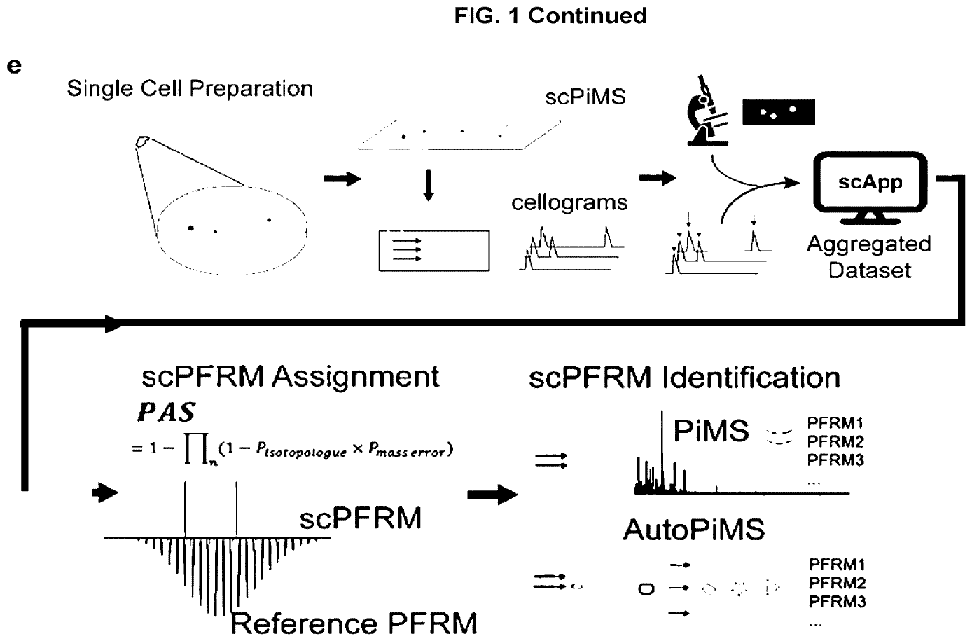

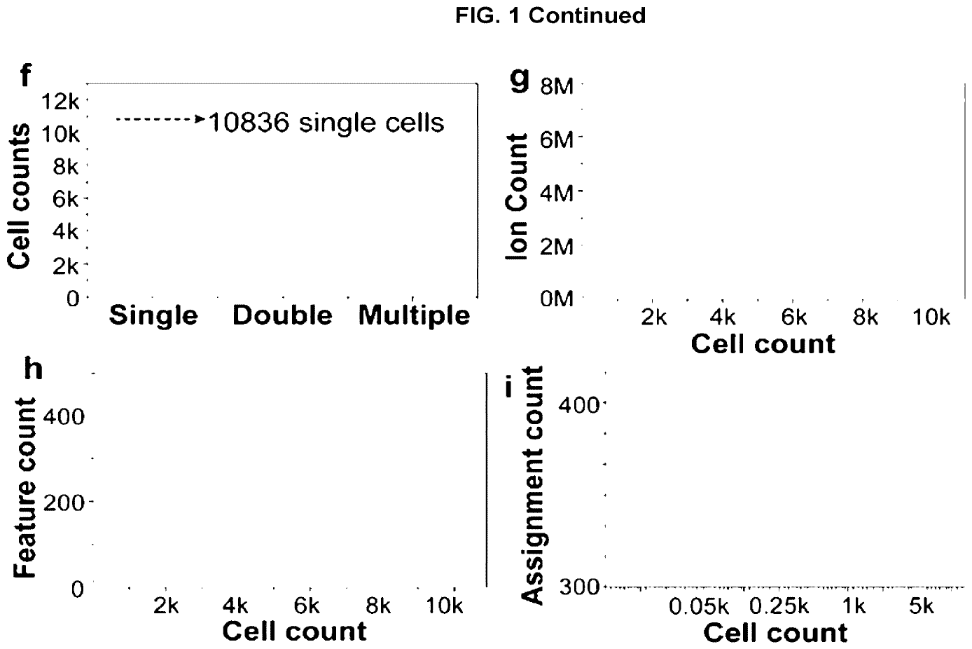

A group of a few thousand cells were disaggregated from a rat hippocampus and drop cast onto a glass slide such that their average spacing was on the order of ~200 μm (FIG. 1a, far left).²⁶ To directly sample proteins from these cells, we employed proteoform imaging²¹ to raster a ~100 μm liquid droplet across the glass slide (FIG. 1a, middle) that is continuously sampled by an electrospray ionization source. As a result, when the droplet reached a single cell (FIG. 1a, middle, image II), thousands of proteoform ions were detected by individual ion mass spectrometry.²⁴ This burst of ions from a single cell was plotted as an extraction curve termed a “cellogram” (FIG. 1a, right), and the 8833 individual proteoform ions collected over six seconds are shown in FIG. 1b.

To expand the number of cells sampled, we used scPiMS at a probe rastering rate of 15 μm/s to scan a slide similar to a PiMS tissue imaging experiment.²¹ The scan rate was set such that the liquid bridge leaves a cell by the time protein extraction wanes (Methods). For a 0.5 cm² region of a slide sampled over ~10 hours, we obtained 586 single cell extraction events with the aggregated cellogram from this run shown in FIG. 1c (Methods). During this acquisition period, 0.72 million individual ions were measured and aggregated (FIG. 1d and Methods). In the aggregated mass spectrum, we used an intact mass tag (IMT) approach to manually identify a few of the most abundant proteoforms by checking against a list of reported rat hippocampal proteoforms within a ±1.5 part-per-million mass tolerance (Methods, Table 1).^{28, 29} The most abundant ones in the aggregated single cell spectrum were isoforms of Myelin basic protein (MBP), High mobility group nucleosome-binding domain 2 (HMGN2), Histones H1 and H2A, Thymosin beta-4 (TYB4), Thymosin beta-10 (TYB10), and ATP synthase subunits.^{28, 29}

The scPiMS 10.8 k Brain Cell Dataset

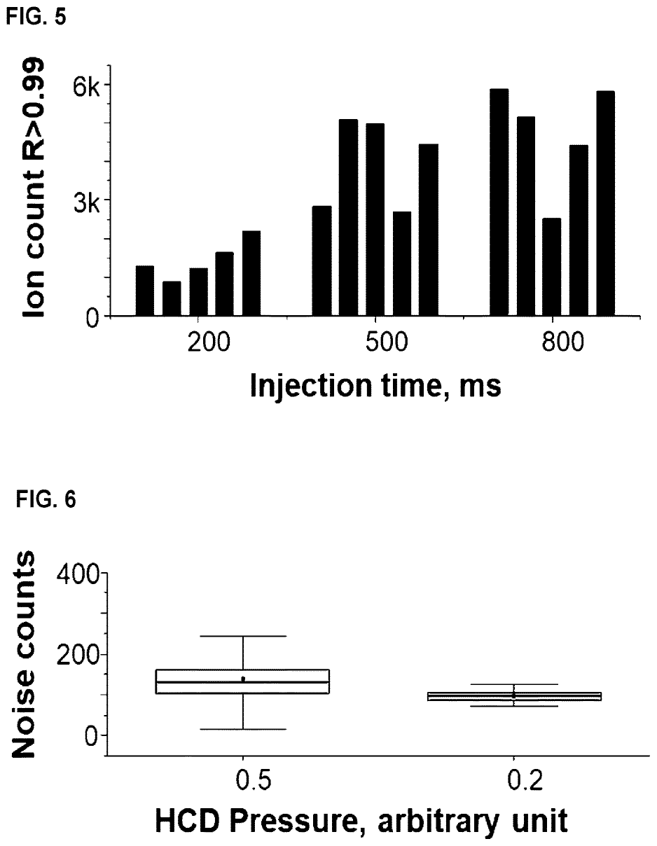

We next used an implementation of scPiMS with ~3-fold higher throughput to examine proteoforms and their distributions across several slides containing a total of ~15 k cells. This experiment took 200 hours of unattended acquisition time using a 200 μm probe at a rastering rate of 40 m/s (i.e., a nominal processing rate of 80 cells/hour). Using the full workflow for scPiMS depicted in FIG. 1e, we detected 12062 cellogram peaks and used an algorithm to extract 10,836 single cell detection events (Methods and FIG. 5). This demonstrated a 90% success rate in converting microscopy-registered features (centered at cell nuclei stained with DAPI) into single cell proteoform detection (FIG. 1f). Upon random spot checking 200 of these cellular detection events, video recorded at 900× magnification during the scPiMS process, all were deemed to be true single cell sampling events.

Assignment of scProteoforms

The 10.8 k dataset was comprised of 48,412 intact ion scans capturing a total of ~16 million raw ions. To assist with assignment of integer charge states to these ions, the dataset was processed by I²MS software¹⁸ after combination with a reference ion library obtained from PiMS imaging of rat hippocampal tissue. The entire dataset for the 10.8 k cells was aggregated together for improved assignment of mass to proteoform isotopic distributions and totaled 7.17 million charge- and mass-assigned individual ions from 5-70 kDa. FIG. 1g shows the rate of accrual for mass-assigned proteoform ions as data were aggregated to 10.8 k cells. The aggregated scPiMS spectrum yielded 472 proteoform features matched to the ion library with a collector's curve for them shown in FIG. 1h. Following this, an application was used to extract ions from single cells (“scApp”, middle panel of FIG. 1e). With thousands of ion masses detected per cell, we developed a statistical framework to assign proteoforms detected in each single cell mass spectrum using a proteoform assignment score (PAS, Methods). This framework and new score assigns a probability that a proteoform is present in a single cell and provides an estimated false discovery rate for this assignment (FDR, see the 4^{th} panel from the left in FIG. 1e and Methods). Using this approach, we obtained an average of 106 scProteoforms detected per cell above 5% estimated FDR.

Identification of scProteoforms

Proteoform identification employed a two-pronged approach (FIG. 1e, far right). We first found 169 putative proteoform IDs using an intact mass tag (IMT) search against ~5,000 candidate proteoforms derived from a group of rat protein sequences transformed from a mouse-hippocampus bottom-up proteomics study using ±1.5 ppm tolerance.³⁰ Next, we obtained another set of experimentally-verified proteoforms obtained using MS/MS mode during PiMS²¹ on brain tissue sections from the same three parent animals in this study (Table 1). From this two-pronged process, we generated a total of 169 identified scProteoforms in this study (data not shown), with 25 of these MS/MS confirmed. Examples are shown in Table 1. The top-three GO terms show enrichment of proteoform detection mapping to key proteins of central metabolism with p-values ranging from 10⁻¹¹ to 10⁻²³. The rate of accrual for already-assigned proteoforms is shown in FIG. 1i. We note that the rate of rise for this collector's curve for proteoform assignment rises far more sharply vs. that for de novo collection of new isotopic distributions (i.e., compare FIG. 1h to FIG. 1i).

scPiMS: Insights into scProteoform Heterogeneity

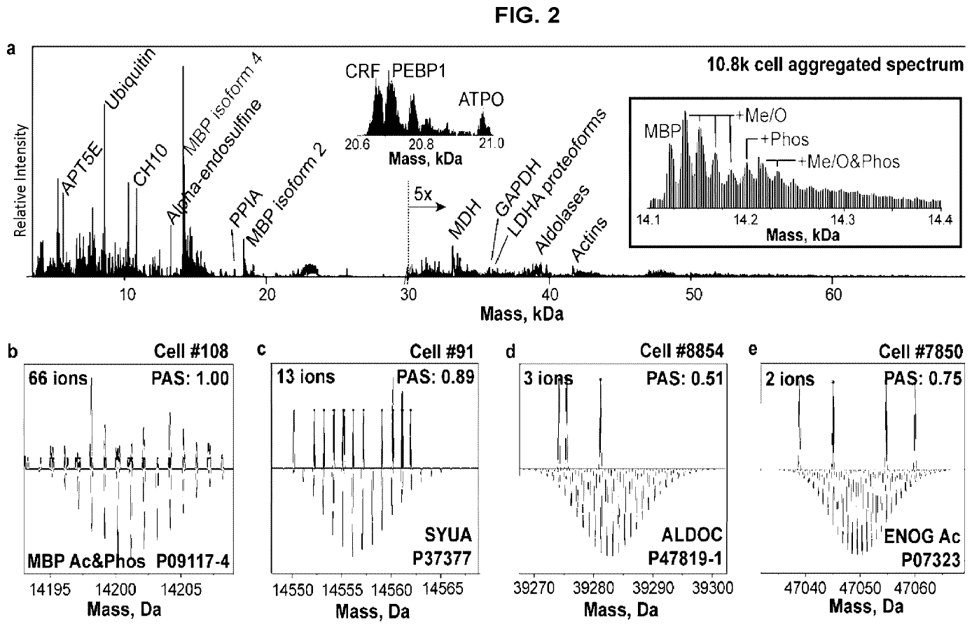

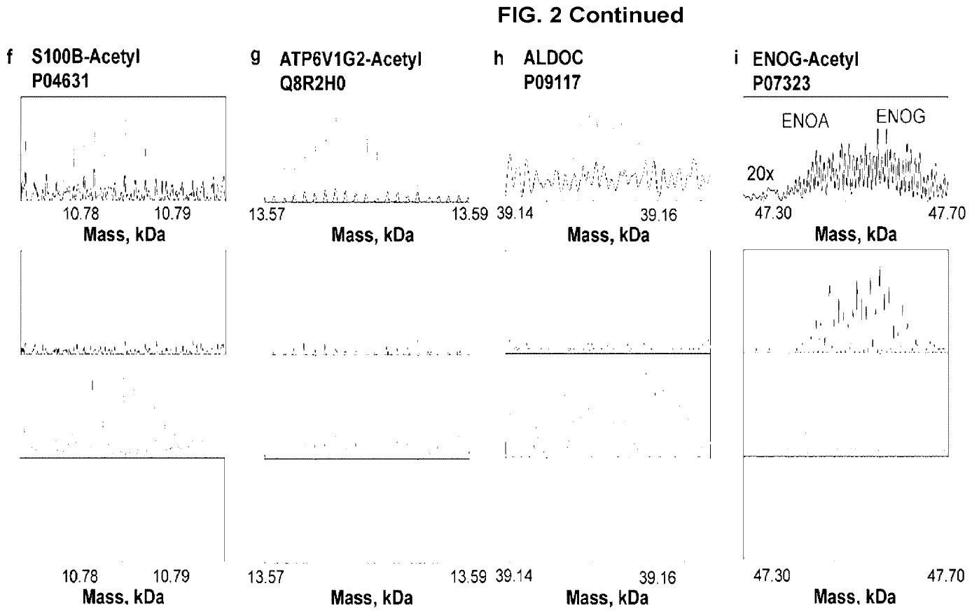

FIG. 2a shows the mass spectrum of the aggregated 10.8 k cell dataset with some of the proteins annotated for which proteoforms were assigned and identified. Myelin basic proteins (MBPs) are among the most abundant in the dataset with a complex landscape of methylation, phosphorylation along with oxidations that can be introduced artefactually in cell processing. A typical MBP scProteoform detection is shown in FIG. 2b containing 66 matched ions and (PAS=1.00). Aside from proteoforms involved in primary metabolism (glycolysis, the TCA cycle, ATP synthase and OXPHOS), we also detected a variety of brain-specific proteoforms including Alpha-endosulfine and Corticoliberin (FIG. 2a). Moreover, we identified several proteoforms that are specifically expressed by cell types in rat hippocampus, in particular, neuronal (e.g., Alpha-synuclein-SYUA, Ubiquitin carboxyl-terminal hydrolase isozyme L1-UCHL1, Gamma-enolase-ENOG) and astrocytic (e.g., Protein S100B, multiple isoforms of Glial fibrillary acidic protein-GFAP, Fructose-bisphosphate aldolase C-ALDOC) proteins. Example scProteoform detection of SYUA, ALDOC, and ENOG cell markers are shown in FIG. 2c-2e with the detection of 2-13 single ions and PAS scores ranging from 0.5-0.9. This highlights the potential of scPiMS in high-throughput cell type classification without the need of antibodies or labels.

We sought next to differentiate major cell types from the individual 10.8 k cells based on spectral features. To this end, a subset of the 13 identified protein markers reported to show differential expression in neurons, astrocytes, and microglia was assembled (e.g., GFAP as a known marker of astrocytes). Candidate cell marker proteoforms were validated against the aggregated single cell spectra, and proteoforms with relatively high abundance, with little or no overlap were selected. We ranked the 10.8 k cells according to a simple binary score for initial assignment of neurons, astrocytes, or microglia (Methods). From the highest scoring subpopulations, we assigned a putative subset of 508 neurons and 1538 astrocytes, and 712 microglia. The aggregated mass spectra from the three cell types show significant differential abundance in four examples of cell marker proteoforms (FIG. 2f-2i).

### Conclusion

A new style of single-cell proteomics was demonstrated on ~10,000 cells with expediency. This work translates the potential of single ion technology into a scalable approach to single-cell proteomics for fast molecular profiling and identification with proteoform specificity up to ~70 kDa. The new scPiMS approach also converts the ‘no-digestion’ or ‘top-down’ philosophy to proteomics into a key advantage that opens a major bottleneck in SCPs by >20-fold (~2000 cells/day compared to ~150 cells/day²⁴), yet involves interpretation of a new data type in the field. This throughput will enable proteoform signatures from rare cells to be captured. As shown here, the scPiMS platform will be advantaged tremendously using a reference set of proteoforms and their ions (c.f, inclusion into the Human Proteoform Atlas³¹) to both accelerate the proteoform annotation step and the depth the analysis obtained in each cell. By providing proteoform-level information, scPiMS promises to advance single cell biology by mapping cell types underlying diverse disease phenotypes. To the extent proteoform measurement connects to complex phenotypes, a mature version of scPiMS may create an outsized impact in single cell biology provided it can find wide adoption in a manner analogous to scRNA-seq.

### Tissue Preparation

Three 2-month-old male Sprague Dawley outbred rats (Rattus norvegicus, Inotiv, West Lafayette, IN) were used in experiments. Animals were fed ad libitum and housed in a 12-h light cycle. Animals were asphyxiated using CO₂. All procedures were performed in accordance with animal use protocol approved by the University of Illinois Institutional Animal Care and Use Committee, and in compliance with both federal and ARRIVE guidelines for the humane treatment of animals.

Immediately after euthanasia, animals were perfused transcardially with ice cold modified Gey's balanced salt solution (mGBSS) containing (in mM): 1.5 CaCl₂), 5 KCl, 0.2 KH₂PO₄, 11 MgCl₂, 0.3 MgSO₄, 138 NaCl, 28 NaHCO₃, and 0.8 Na₂HPO₄, and 25 HEPES, pH 7.2. One third of the hippocampus from one hemisphere was surgically dissected for single cell preparation. Intact hemispheres of the same brains as used for cell population collection were frozen and sectioned for intact tissue PiMS imaging. Rat hippocampus tissue punches and thin sections were prepared according to published protocols.³² Rat brain hemisphere slices 16 m thick were cut at −16 °C using a cryostat (Leica CM3050 S, Leica Biosystems GmbH, Wetzlar, Germany). Brain slices containing hippocampal areas of all three animals were deposited on the same slide. Sections were rinsed with 200 Proof ethanol twice. Tissue samples were stored at −80 °C before imaging analysis by scPiMS using the published approach.²¹

### Preparation of Individual Cell Populations.

We used the procedures for single cell isolation developed for single cell metabolomics, adapted here for scPiMS.^{26, 33} One-third of the hippocampus from one hemisphere was surgically dissected and treated with the papain dissociation system (Worthington Biochemical, Lakewood, NJ) for 120 min. at 34 °C with superficial oxygenation. After the treatment, tissues were gently rinsed twice with ice cold mGBSS. Mechanical tissue dissociation was performed in ice cold mGBSS supplemented with Hoechst 33342 (1 μg/mL). The individual cell suspension was deposited onto indium tin oxide (ITO) glass slides (Delta Technologies, Loveland, CO) to achieve low density cell population with individual cells spaced by an average of >200 um. Cell populations from three different animals were deposited onto separate marked areas on ITO glass slides. Cells were sedimented and adhered to slide surface for 10-20 min. Surrounding cell media was quickly replaced with mGBSS containing 33% glycerol. After brief incubation for ~10-20 s most of the solution present on ITO glass slide was removed. Slides were rinsed twice with 200 Proof ethanol (Lab Alley, Spicewood, TX).

### Fluorescent Microscopy Experiments

Images of populations of single cells were taken in mixed bright-field and fluorescence mode using a Zeiss Axio M2 microscope (Zeiss GmbH, Jena, Germany) equipped with an AxioCam ICc5 camera, X-cite Series 120 Q mercury lamp (Lumen Dynamics, Mississauga, ON, Canada) and a HAL 100 halogen illuminator (Zeiss). The DAPI (ex. 335-383 nm; em. 420-470 nm) dichroic filter was used for fluorescence excitation. A 2.5× objective was used for fast acquisition with a 13% overlap between individual images allowing proper image stitching and formation of a single view for entire glass slide. Images were processed and exported as big tiff files using ZEN software version 2 blue edition (Zeiss). Numbers of cells on slide in regions of interest were determined using ImageJ.³⁴

scPiMS Probe Fabrication and Ion Source Conditions

A custom-designed nano-DESI source was used for all data acquisition. The experimental details of nano-DESI MSI have been described elsewhere.^{31, 36} Briefly, the nano-DESI probe is comprised of a flame-pulled fused silica primary (OD 40 μm, ID 20 m, Molex, Thief River Falls, MN) and a nanospray capillary (OD 150 μm, ID 40 m) with the spray side of the nanospray capillary positioned towards the MS inlet. The nano-DESI probe utilizes a dynamic liquid bridge formed between the capillary junction and the glass surface to extract analytes when brought into contact with the glass surface. The liquid bridge is dynamically maintained by solvent propulsion from the primary capillary and instantaneous vacuum aspiration through the nanospray capillary. All samples were electrosprayed in positive ion mode under denaturing conditions in a 60%/39.4% acetonitrile/water and 0.6% acetic acid solution compatible with both protein extraction and ionization. The solvent flow rate was kept in the range of 160-500 nL/min. The ion source conditions on the mass spectrometer were set as follows: ESI voltage: +1.7-3 kV; in-source CID: 15 eV; S-lens RF level: 80%; capillary temperature: 360 °C.

scPiMS Operation Modes

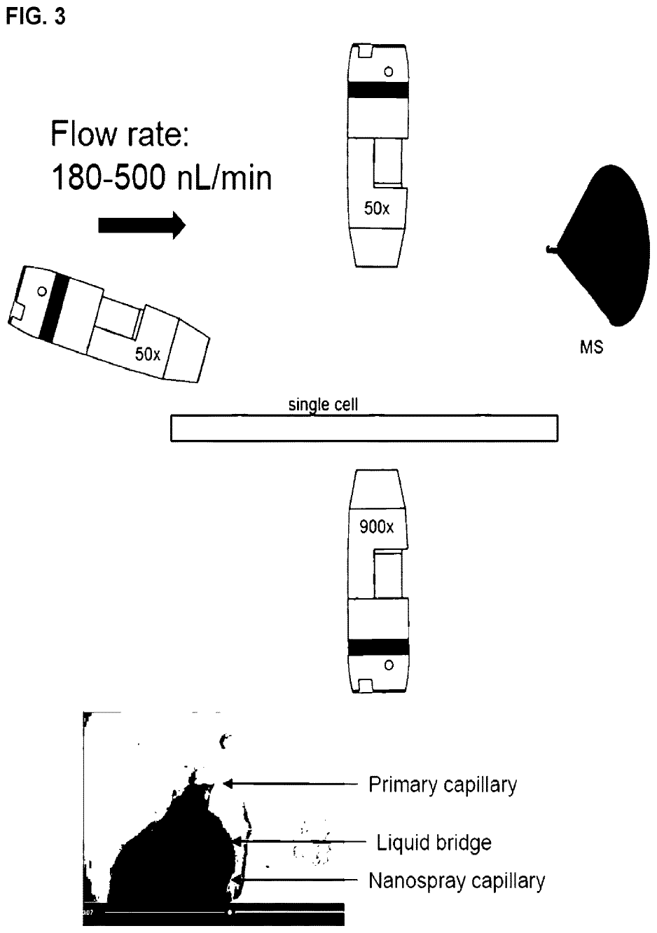

scPiMS experiments were performed in two distinct modes: targeted mode and high-throughput scanning mode. In targeted mode, a single cell feature was first found by the 900× high-magnification microscope (FIG. 3). In particular, a nano-DESI probe was brought into contact with the glass surface at a location ~50 m away from the single cell to form the liquid bridge, and was subsequently moved toward the cell with manually-controlled stage motions after MS data acquisition was triggered. The probe was typically parked on the cell for one minute to obtain a targeted “cellogram” (chronogram of total ion counts). The cellogram typically starts from a drastic rise in protein signal followed by an exponential decay over time, which is characteristic of the extraction kinetics of cellular protein analyzed by scPiMS.

High-throughput scanning mode was performed by moving the glass slide under the nano-DESI probe in parallel lines at a constant linear velocity. The spacing between adjacent lines was set similar to the size of the liquid bridge formed by the probe and the surface to ensure all the surface area containing single cells were covered. The size of the liquid bridge in nano-DESI can be adjusted in range from 25 to 400 μm depending on the size of the capillaries used to fabricate the probe. For scPiMS, 200 m was selected considering the throughput of the experiment to covering the entire area containing single cells. Larger probes will result in multiple adjacent cells on the surface being analyzed at the same time, which obscures the protein profiles from these individual single cells. Statistical modeling was carried out to simulate the percentage of single cell extraction events using the single cell coordinates obtained from fluorescence microscopy. In particular, surface area containing single cells was divided into 200 m square grids, and number of grids that contain one cell or more than one cell were calculated. At the density of cells loaded on the glass slides, 90% of the cells can be sampled as single cell features using the 200 μm probe (FIG. 1f).

### Cellogram Optimization

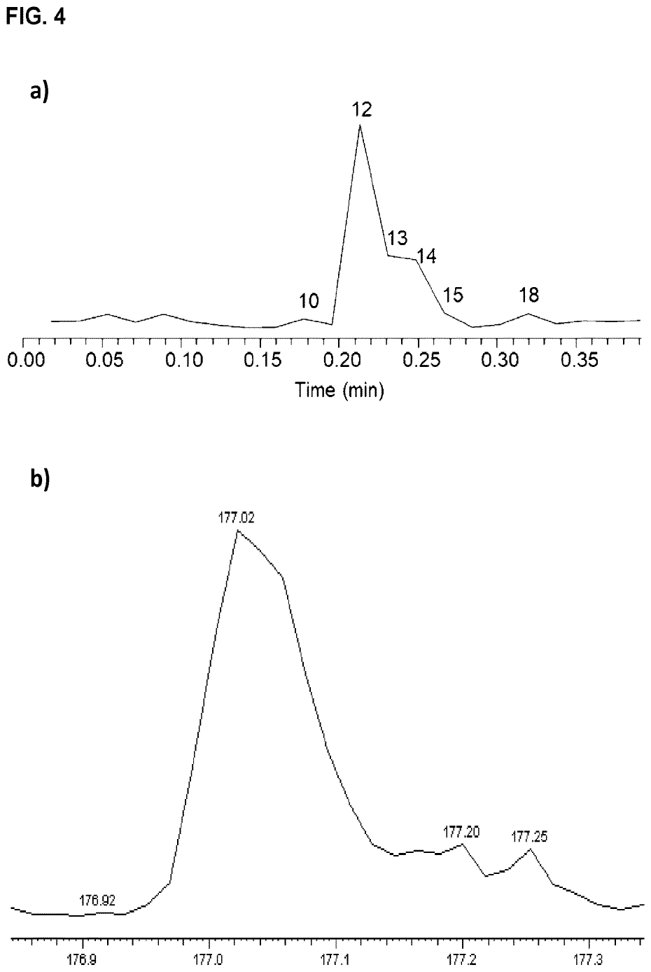

A cellogram obtained in targeted mode revealed the extraction kinetics of scPiMS probe on single cells and provided guidance to optimizing the parameters in high-throughput scanning mode. In particular, a targeted mode cellogram was obtained by parking the probe at the cell and monitoring the total ion response. The time at which the targeted cellogram falls back to the baseline indicated that readily extractable proteins were captured. This gave the approximate time that the cell should be exposed to the probe and informs the overall throughput for scPiMS. More importantly, in high throughput scanning experiments, extraction of protein content from a single cell before getting in contact with the next cell guarantees that the signal carryover between adjacent cells is minimized. The upper limit was employed to calculate the “exposure time” of a single cell to the liquid bridge in the rastering mode. The average exposure time for single cells at a 500 nL/min. solvent flow rate was found to be 5 s from a few targeted cellograms (FIG. 4). The rastering scan rate was determined by the size of the liquid bridge, size of the single cell, and the exposure time (Eq. 1). Considering the small size of the rat brain single cells (~10 μm) compared to the liquid bridge (200 μm) in the current scPiMS configuration, the size of the liquid bridge was used to calculate the rastering scan rate (Eq. 1):

$$Rastering scan rate = \frac{Probe size + single cell size}{Exposure time} ≅ \frac{Probe size}{Exposure time} \qquad ( Eq . 1 )$$

A 40 m/s scan rate (200 m divided by 5 s) was used for all the 10.8 k scPiMS runs. Interestingly, the exposure time was found to be flow-rate dependent. In particular, in precursor 2 k single cell runs, a reduced solvent flow rate at 180 nL/min was employed, which gave an average exposure time of 8 s (FIG. 4). We reasoned that the solvent replenishing rate and band diffusion in the liquid flow in the secondary capillary both contribute to the observed slower kinetics of extraction. This further suggests that targeted cellograms should be obtained before starting the high-throughput scanning experiments if scPiMS sampling conditions were changed.

scPiMS Data Acquisition

scPiMS data acquisition was performed in the individual ion mass spectrometry (I²MS) mode on a previously described Orbitrap Q-Exactive Plus mass spectrometer (Thermo Fisher Scientific GmbH, Bremen, Germany).²⁴ The spectral acquisition rate was set at one scan per second. During scPiMS data acquisition, proteoforms from single cells were sampled and ionized by a nano-DESI probe to generate multiply-charged ions distributed across multiple charge states. The ion injection time was optimized such that ions in one detection period were collected in the individual ion regime, which gives a singular ion signal at a defined m/z (or frequency) value. Due to the protein signals emerging from single cells, the majority of the scPiMS ion detection periods were dominated by individual ion signals even at extended MS injection time up to 800 ms. Despite the longer injection time allowed for collection of more ions in the C-trap and the Orbitrap analyzer, we observed higher chemical noise and loss of trapping efficiency as trade-offs. Targeted mode scPiMS experiment was employed to optimize the injection time up to 800 ms. An ion injection time of 500 ms gave the best performance in terms of signal to noise and was used for all experiments unless otherwise specified (FIG. 5).

As discussed in previous work, the HCD pressure level has a strong impact on the ion accumulation and survival performance in I²MS. The HCD pressure level was typically set at 0.5 (UHV pressure <5×10⁻¹¹ Torr) to reduce collision-induced ion decay within the Orbitrap analyzer without sacrificing the trapping efficiency for intact protein ions. The HCD pressure setting of 0.2 was used throughout the scPiMS experiments to reduce the chemical noise collected in the spectrum at extended MS injection times (FIG. 6).

In addition, the Orbitrap central electrode voltage was adjusted to −1 kV to improve the ion survival rate in I²MS. Additional relevant data acquisition parameters were as follows: mass range: 500-1500 n z; AGC mode: disabled; enhanced Fourier transform: off, averaging: 0; microscans: 1. Time-domain data files were acquired at detected ion frequencies and recorded as Selective Temporal Overview of Resonant Ions (STORI) files.³⁷ STORI setting: Enabled.

scPiMS Feature Selection & Data Analysis

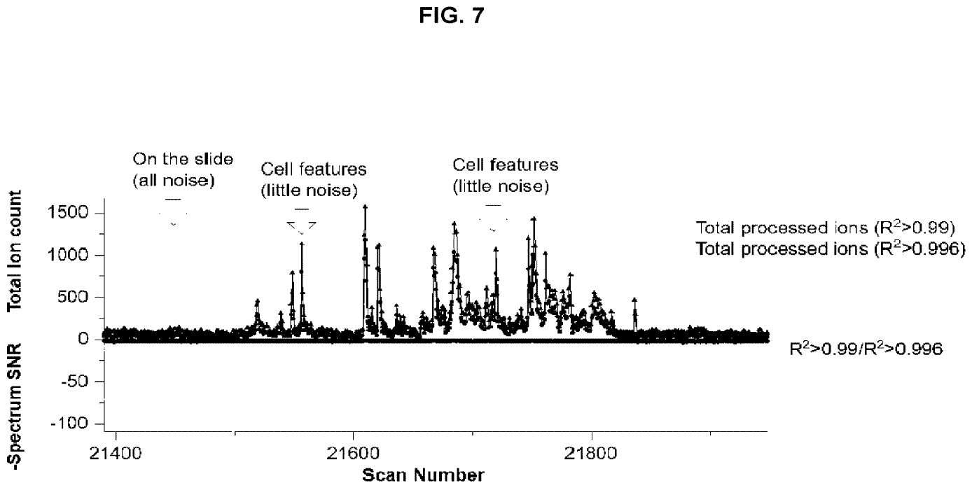

scPiMS data analysis was performed using MATLAB and C^{#} script developed in-house. scPiMS raw data was collected as continuous chronograms containing discrete or overlapping “cellograms” and other peak features from chemical noise on the glass slide collected during the experiments. Cellogram picking was first performed to recognize MS scans corresponding to true single cell events and eliminate the blank and noise scans from the dataset. We first took advantage of STORI analysis in I²MS processing to stratify the “protein-signal-like” and “noise-like” scans. In particular, typical chemical-noise-like ions from the ion source show lower ion lifetime and lower R² in their STORI plots. We found the R² cutoff of 0.999 as a suitable threshold to achieve sufficient signal-to-noise ratio for extraction of true single cellograms (FIG. 7).

The ion chronogram constructed at R²>0.999 was employed for peak picking with a peak intensity threshold of 10 and a minimum peak distance of 5 (exposure time*MS scan rate). In specific cases, the threshold was set to 50 and the R² cutoff was set to 0.9996 to take into account the variation of the noise level in the dataset on different days of acquisition when instrument performance changed notably. The peak features picked by the program were further categorized into single-cell multi-cell and cellogram-like features using peak characteristics. In particular, if the half-width-at-half-maximum on the right side of a cellogram was more than two scans (2 s), the feature was recognized as a multi-cell feature. Multi-cell features were strongly filtered in this step and only the single cell features were used for further analysis. The number of single cell features was also validated and matched to the fluorescent single cell feature count as described in the “scPiMS operation modes” section. We observed in rare cases that the single cell feature count is substantially lower than the simulated fluorescent count. This was attributed to aberrant probe conditions, and the peak picking criteria were not further adjusted to match the fluorescent features. From a total of 15.1 k single cell features, we were able to detect 10.8 k single-cell-like features over the course of 10 days.

The STORI files of the MS scans from the single-cell-like features were reextracted from the dataset and concatenated to construct a mass-domain spectrum. In particular, all individual ion signals were resubjected to charge state assignment for mass calculation. The neutral masses of the protein ions were calculated by:

$$Mass = ( \frac{m}{z} \times z ) - ( z + {M}_{proton} ) \qquad ( 2 )$$

Charge state (z) is obtained from the slope of induced image current determined by the STORI analysis described in detail elsewhere.³⁷ Accurate charge assignment of each ion was statistically evaluated by comparing the slopes of its isotopologues across different charge states from the entire dataset. We employed a probability metric to filter out ions with a lower probability score and construct mass-domain isotopic distribution with statistical confidence.
scProteoform Scoring and False Discovery Rate (FDR)

Individual ions corresponding to each single cell feature were aggregated into a single cell spectrum and were utilized to generate a score for each proteoform in a given cell feature. The proteoform assignment space may be determined via multiple pathways; either isotopic envelopes created via the THRASH deconvolution algorithm³⁸ or envelopes created and validated through identified proteoforms from intact mass tag (IMT) search or MS/MS via the PiMS platform described in detail in the “Intact Mass Tag (IMT) Search & Gene Ontology (GO) analysis” and “Data analysis and Proteoform MS/MS along with Identification” section. Cell specific individual ions were searched against the isotopic envelope library and matched to specific isotopic peaks with a tolerance of ±0.3 Da, these ion matches are scored and combined with all ion scores for a given proteoform to yield a single cell proteoform score (PAS) using the following equation:

$$PAS = 1 - {\prod }_{k = 1}^{n} 1 - ( {P}_{isotopologue} \times {P}_{mass error} ) \qquad ( 3 )$$

Where P_{isotopologue} is the expected relative intensity of the matching isotopologue and P_{mass error} is a probability score for the mass error between the observed individual ion mass and the theoretical mass of the isotopologue, utilizing the cumulative distribution function (CDF) of a normal distribution with a mean and sigma determined by the theoretical mass and width of a isotopic peak respectively:

$${P}_{mass error} = \{ \begin{matrix}CDF ( {m}_{ion} ) & {m}_{ion} < {m}_{iso} \\ 1 - CDF ( {m}_{ion} ) & {m}_{ion} > {m}_{iso}\end{matrix} \qquad ( 4 )$$

Where mᵢₒₙ is the mass of the individual ion and mᵢₛₒ is the theoretical mass of the matched isotopologue. To perform multiple hypothesis testing, an empirical false discovery rate (FDR) procedure was implemented at the single cell level. Decoy proteoforms (10 per proteoform in the library) were generated using random amino acids and constrained to be the same sequence length as the representative proteoform. These decoys were scored alongside the proteoform hits, rank ordered and all proteoform hits given a q-value. Proteoforms with a q-value less than 0.05 (5% FDR) were classified as assigned in that single cell.

### Cell Type Marker Scores

Using the FDR controlled proteoforms scores for various cell type markers for astrocytes and neurons, we were able to create a cell type score for each cell. Marker proteoform scores associated with a given cell type were combined with the following:

$${Score}_{cell type} = 1 - {\prod }_{k = 1}^{n} ( 1 - PAS ) \qquad ( 5 )$$

Where n is the number of markers used for the particular cell type, Normalization factor is the inversed abundance ratio of the marker with the least abundant cell marker in that type in the ion library. The scoring system takes into account the contribution of different markers ion the ion library in the charge assignment process and treats each marker as equally effective to define the cell type.

### Intact Mass Tag (IMT) Search & Gene Ontology (GO) Analysis

The summed mass-domain ion library was converted to .mzML format and processed using a custom version of TD Validator (Proteinaceous, Evanston, IL) implemented with an MS¹ IMT search function. The library spectrum was self-calibrated by +10.25 ppm according to the accurate masses of six identified proteoforms in the 10-50 kDa mass range. A custom protein database was constructed from top 1000 most abundant proteins in a bottom-up proteomics study of mouse brain hippocampus was used for the search (data not shown).³⁹ In particular, the protein names of the top 1000 mouse proteins were transformed into the corresponding rat proteins that resulted in 764 hits.³⁹ The IMT search was performed with a ±1.5 ppm mass tolerance considering Methionine on/off, monoacetylation, and monophosphorylation as possible proteoform modifications in the database (~5000 proteoforms search space). Additional proteoform matches were curated by spectrum inspection and manual annotation of putative modifications recorded in the hippocampal-specific top-down proteomics study by Fournier et al.²⁸ and the Swiss-Prot rat proteome database, resulting in a total of 169 proteoform matches.

Gene Ontology (GO) analysis using Metascape (metascape.org) was performed by retrieving a list of Entrez Gene ID for the 169 identified proteoforms using the ID mapping tool on UniProt.⁴⁰ The result contains the top-level GO biological processes.

Data Analysis for Proteoform Identification by MS/MS Along with Identification

Targeted MS/MS experiments were performed on a tissue section adjacent to the imaged section using higher-energy collisional dissociation (HCD). In the first step, a target proteoform was selected in the mass-domain spectrum. We utilized the PiMS image of the target proteoform to select a target area on the section where the proteoform abundance is enhanced. For the selected area, the mass-domain PiMS spectrum was convoluted back to m z domain, from which a proper isolation window that contains predominantly the target proteoform was selected. A 0.8 mi z isolation window was typically employed for most of the targets; in special cases, 0.5-0.6 m z window was used to avoid overlapping signal. MS/MS experiments were performed by scanning the nano-DESI probe over the selected region with the selected isolation window at 2.5-4 μm/s scan rate. MS/MS data acquisition was conducted in the I²MS mode with an Orbitrap detection period of 2 s (HCD pressure setting=0.5).⁴¹ HCD collision energy and injection time was optimized to maximize the population of individual ion fragments. Typical ranges of collision energy and injection time used in this study were 7-14 eV and 200-1500 ms, respectively. Total data acquisition time for each target varied from 1-5 hours.

MS/MS data were first subjected to I²MS processing for fragment ion charge assignment and mass-domain spectrum construction following the same procedure as described above. Mass-domain spectrum was converted to .mzML format subjected to MS² search function implemented in ProSight Native (Proteinaceous, Evanston, IL) to look for possible candidates from the entire human protein database. For each search, the top 1-5 candidates were manually validated using TDValidator (Proteinaceous) to identify the best matching proteoform. When two top candidates of similar sequence coverage were found for one MS/MS experiment, both were reported. Proteoform E-values were obtained from ProSight Native and TD Validator reports.

TABLE 1
Proteoforms identified from scPiMS and proteoform imaging of rat hippocampus.

| | | | | | Mass | |
|---|---|---|---|---|---|---|
| Protein | | | | Theoretical | accuracy, | |
| description | Gene | Accession | PFR | mass | ppm | Modifications |
| Glial fibrillary | GFAP | P47819 | N/A | 49969.61 | 0.65 | Acetyl@N |
| acidic protein | | | | | | |
| Gamma- | ENOG | P07323 | N/A | 47022.89 | 0.16 | Met-off, |
| enolase | | | | | | acetyl@N |
| Alpha-enolase | ENOA | P04764 | N/A | 47010.22 | 0.99 | Met-off, |
| | | | | | | acetyl@N |
| Alpha- | SYUA | P37377 | N/A | 14549.21 | 0.94 | Met-off, |
| synuclein | | | | | | acetyl@N |
| Myelin basic | MBP | P02688 | N/A | 14194.16 | 0.48 | Met-off, |
| protein | | | | | | acetyl@N, |
| isoform 4 | | | | | | phosphoryl@S |

### REFERENCES

1. Smith, L. M. & Kelleher, N. L. Proteoforms as the next proteomics currency. Science 359, 1106-1107 (2018).
2. Melani, R. D. et al. The Blood Proteoform Atlas: A reference map of proteoforms in human hematopoietic cells. Science 375, 411-418 (2022).
3. Gawad, C., Koh, W. & Quake, S. R. Single-cell genome sequencing: current state of the science. Nature Reviews Genetics 17, 175-188 (2016).
4. Tang, F. et al. mRNA-Seq whole-transcriptome analysis of a single cell. Nat. Methods 6, 377-382 (2009).
5. Jaitin Diego, A. et al. Massively Parallel Single-Cell RNA-Seq for Marker-Free Decomposition of Tissues into Cell Types. Science 343, 776-779 (2014).
6. Burnum-Johnson, K. E. et al. New Views of Old Proteins: Clarifying the Enigmatic Proteome. Mol. Cell. Proteomics 21, 100254 (2022).
7. Dou, M. et al. High-Throughput Single Cell Proteomics Enabled by Multiplex Isobaric Labeling in a Nanodroplet Sample Preparation Platform. Anal. Chem. 91, 13119-13127 (2019).
8. Budnik, B., Levy, E., Harmange, G. & Slavov, N. SCoPE-MS: mass spectrometry of single mammalian cells quantifies proteome heterogeneity during cell differentiation. Genome Biol. 19, 161 (2018).
9. Piehowski, P. D. et al. Automated mass spectrometry imaging of over 2000 proteins from tissue sections at 100-μm spatial resolution. Nat. Commun. 11, 8 (2020).
10. Zhu, Y. et al. Nanodroplet processing platform for deep and quantitative proteome profiling of 10-100 mammalian cells. Nat. Commun. 9, 882 (2018).
11. Claudia, C. et al. An automated workflow for multiplexed single-cell proteomics sample preparation at unprecedented sensitivity. bioRxiv, 2021.2004.2014.439828 (2022).
12. Aebersold, R. & Mann, M. Mass-spectrometric exploration of proteome structure and function. Nature 537, 347-355 (2016).
13. Matzinger, M., Mayer, R. L. & Mechtler, K. Label-free single cell proteomics utilizing ultrafast LC and MS instrumentation: A valuable complementary technique to multiplexing. Proteomics n/a, 2200162 (2023).
14. Klont, F. et al. Assessment of Sample Preparation Bias in Mass Spectrometry-Based Proteomics. Anal. Chem. 90, 5405-5413 (2018).
15. Marx, V. A dream of single-cell proteomics. Nat. Methods 16, 809-812 (2019).
16. Smith, R. D. Large Individual Ion FTICR Measurements from the Mid-1990s Using Reactions for Charge Determination Mass Spectrometry. Journal of the American Society for Mass Spectrometry 34, 803-812 (2023).
17. Makarov, A. & Denisov, E. Dynamics of ions of intact proteins in the Orbitrap mass analyzer. Journal of the American Society for Mass Spectrometry 20, 1486-1495 (2009).
18. Kafader, J. O. et al. Measurement of Individual Ions Sharply Increases the Resolution of Orbitrap Mass Spectra of Proteins. Anal. Chem. 91, 2776-2783 (2019).
19. Valaskovic, G. A., Kelleher, N. L., Little, D. P., Aaserud, D. J. & McLafferty, F. W. Attomole-Sensitivity Electrospray Source for Large-Molecule Mass Spectrometry. Anal. Chem. 67, 3802-3805 (1995).
20. Seeley, E. H. & Caprioli, R. M. Molecular imaging of proteins in tissues by mass spectrometry. Proc. Natl. Acad. Sci. 105, 18126-18131 (2008).
21. Su, P. et al. Highly multiplexed, label-free proteoform imaging of tissues by individual ion mass spectrometry. Science Advances 8, eabp9929 (2022).
22. Worner, T. P. et al. Resolving heterogeneous macromolecular assemblies by Orbitrap-based single-particle charge detection mass spectrometry. Nat. Methods 17, 395-398 (2020).
23. Zubarev, R. A. & Makarov, A. Orbitrap mass spectrometry. Anal. Chem. 85, 5288-5296 (2013).
24. Kafader, J. O. et al. Multiplexed mass spectrometry of individual ions improves measurement of proteoforms and their complexes. Nature methods 17, 391-394 (2020).
25. McGee, J. P. et al. Isotopic Resolution of Protein Complexes up to 466 kDa Using Individual Ion Mass Spectrometry. Anal. Chem. 93, 2723-2727 (2021).
26. Neumann, E. K., Comi, T. J., Rubakhin, S. S. & Sweedler, J. V. Lipid Heterogeneity between Astrocytes and Neurons Revealed by Single-Cell MALDI-MS Combined with Immunocytochemical Classification. Angew. Chem. Int. Ed. 58, 5910-5914 (2019).
27. Shalek, A. K. et al. Single-cell transcriptomics reveals bimodality in expression and splicing in immune cells. Nature 498, 236-240 (2013).
28. Delcourt, V. et al. Spatially-Resolved Top-down Proteomics Bridged to MALDI MS Imaging Reveals the Molecular Physiome of Brain Regions*. Mol. Cell. Proteomics 17, 357-372 (2018).
29. Hanrieder, J., Wicher, G., Bergquist, J., Andersson, M. & Fex-Svenningsen, A. MALDI mass spectrometry based molecular phenotyping of CNS glial cells for prediction in mammalian brain tissue. Analytical and Bioanalytical Chemistry 401, 135-147 (2011).
30. Wang, D. et al. A deep proteome and transcriptome abundance atlas of 29 healthy human tissues. Mol. Syst. Biol. 15, e8503 (2019).
31. Hollas, M. A. R. et al. The Human Proteoform Atlas: a FAIR community resource for experimentally derived proteoforms. Nucleic Acids Res. 50, D526-D533 (2022).
32. Neumann, E. K. et al. Protocol for multimodal analysis of human kidney tissue by imaging mass spectrometry and CODEX multiplexed immunofluorescence. STAR Protocols 2, 100747 (2021).
33. Neumann, E. K., Ellis, J. F., Triplett, A. E., Rubakhin, S. S. & Sweedler, J. V. Lipid Analysis of 30 000 Individual Rodent Cerebellar Cells Using High-Resolution Mass Spectrometry. Anal. Chem. 91, 7871-7878 (2019).
34. Schneider, C. A., Rasband, W. S. & Eliceiri, K. W. NIH Image to ImageJ. 25 years of image analysis. Nat. Methods 9, 671-675 (2012).
35. Yin, R., Burnum-Johnson, K. E., Sun, X., Dey, S. K. & Laskin, J. High spatial resolution imaging of biological tissues using nanospray desorption electrospray ionization mass spectrometry. Nat. Protoc. 14, 3445-3470 (2019).
36. Laskin, J., Heath, B. S., Roach, P. J., Cazares, L. & Semmes, O. J. Tissue imaging using nanospray desorption electrospray ionization mass spectrometry. Anal. Chem. 84, 141-148 (2012).
37. Kafader, J. O. et al. STORI plots enable accurate tracking of individual ion signals. Journal of The American Society for Mass Spectrometry 30, 2200-2203 (2019).
38. Horn, D. M., Zubarev, R. A. & McLafferty, F. W. Automated reduction and interpretation of high resolution electrospray mass spectra of large molecules. Journal of the American Society for Mass Spectrometry 11, 320-332 (2000).
39. Sharma, K. et al. Cell type- and brain region-resolved mouse brain proteome. Nat. Neurosci. 18, 1819-1831 (2015).
40. Zhou, Y. et al. Metascape provides a biologist-oriented resource for the analysis of systems-level datasets. Nat. Commun. 10, 1523 (2019).
41. Kafader, J. O. et al. Individual ion mass spectrometry enhances the sensitivity and sequence coverage of top-down mass spectrometry. J. Proteome Res. 19, 1346-1350 (2020).
42. Kafader, J. O. et al. Multiplexed mass spectrometry of individual ions improves measurement of proteoforms and their complexes. Nat. Methods 17, 391-394 (2020).

### Example 2

Single-cell proteomics (SCP) seeks to capture the heterogeneity in cell type, state and function obscured by population-averaged protein measurements. Biology at single cell resolution enables characterization of normal and pathological states of cells comprising complex tissues.¹ Established methods for single cell proteomics using antibody-based molecular probes require prior knowledge to protein targets and lack the specificity to discover or measure proteoforms,² the exact molecular forms of proteins present in different cell types.

Combining the molecular specificity of proteoform analysis with single cell measurement presents major challenges. Unlike amplification-based genomics platforms for single-cell RNA sequencing (scRNA-seq),³ SCP relies upon a direct detection paradigm to interrogate the complexity of the proteome.⁴ Mass spectrometry (MS) based ‘bottom-up’ platforms such as Deep Visual Proteomics,⁵ Nanodroplet processing in one-pot for trace samples (nanoPOTS)⁶ and Single Cell ProtEomics by Mass Spectrometry (SCoPE-MS)⁷ can identify and quantify thousands of proteins.⁸ Despite significant advances, the rate of single cell processing is commonly a few hundred per day, even from cell lines.^{9, 10} To increase throughput, avoid multi-step processing and protein digestion, a more direct sampling approach could be advantageous for ‘top-down’ proteoform detection in single cells.¹¹ However, a scheme for MS-based intact proteoform SCP sets requirements for sensitivity and protein identification that have yet to be met.^{12, 13}

Here, we introduce single-cell Proteoform imaging Mass Spectrometry (sePiMS) for sampling of proteoform molecules directly from single cells. This new approach utilizes nanospray desorption electrospray ionization (nano-DESI), a sensitive localized liquid-extraction surface sampling approach widely applied for MS imaging.^{11, 14, 15} Nano-DESI is coupled with individual ion MS (I²MS), a new technique for single ion detection with >500× greater sensitivity and 10× higher resolving power over traditional Orbitrap spectra.^{16, 17} Recently, this combination captured the small bursts of highly localized proteoforms¹⁸ up to ~70 kDa encountered in tissue imaging of thin sections at ~20 mm spatial resolution.^{19, 20} Here we demonstrate the ability of scPiMS for proteoform extraction and MS detection of single NIH 3T3 cells at a raw rate of <10 seconds/cell. We detected hundreds of proteoforms and identified 41 of them from 38 abundant proteins on a slide containing ~500 single cells that were verified using optical microscopy.^{21, 22} scPiMS demonstrates a high potential to advance single cell biology by increasing throughput, analogous to the very early days of scRNA-seq.²³

### Preparation of NIH 3T3 Cells on Glass Slide

NIH 3T3 mouse embryonic fibroblast cells were used as standard cells for optimization of sample preparation conditions, solvent conditions and instrument parameters in scPiMS. NIH 3T3 cells stored in liquid nitrogen were thawed in a cryovial at 37 °C water bath and resuspended in room-temperature 1× phosphate-buffered saline (PBS). The cells were cultured in a T75 flask using Dulbecco's Modified Eagle Medium (DMEM) with an addition of 10% fetal bovine serum (FBS) and 1% penicillin-streptomycin at 37 °C in a CO₂ incubator with 5% CO₂. The cells were harvested from the flask after 24 h by the addition of trypsin-EDTA 0.25% for detachment from the bottom of the cell culture flask. The cells were spun down into a loose pellet by a centrifuge at 1,000 g and washed by 1×PBS twice. The number of cells in the final suspension was determined by a Nexcelom Auto T4 cellometer (Nexcelom Bioscience, Lawrence, MA). The cell suspension was diluted to a concentration of 5 k/mL by 1×PBS and drop cast into a Nunc Lab-Tek II chamber slide (ThermoFisher Scientific, Waltham, MA) and incubated for 5-10 minutes at 37 °C in a CO₂ incubator. In the control fixation protocol, the 1×PBS buffer was slowly removed by vacuum and the cells adhered to the slide were subsequently fixed by 200 proof cold ethanol (−20 °C) prior to scPiMS analysis. Other fixation protocols tested were summarized in FIG. 18.

scPiMS Probe Fabrication and Ion Source Conditions

A custom-designed nano-DESI source was used for all data acquisition. The experimental details of nano-DESI MSI have been described elsewhere.^{14, 15} Briefly, the nano-DESI probe is comprised of a flame-pulled fused silica primary (OD 40 m, ID 20 m, Molex, Thief River Falls, MN) and a nanospray capillary (OD 150 μm, ID 40 μm) with the spray side of the nanospray capillary positioned towards the MS inlet. The nano-DESI probe utilizes a dynamic liquid bridge formed between the capillary junction and the glass surface to extract analytes when brought into contact with the glass surface. The liquid bridge is dynamically maintained by solvent propulsion from the primary capillary and instantaneous vacuum aspiration through the nanospray capillary. All samples were electrosprayed in positive ion mode under denaturing conditions in a 60%/40% acetonitrile/water with the addition of 2% glacial acetic acid compatible with both protein extraction and ionization. The solvent flow rate was kept at 500 nL/min. The ion source conditions on the mass spectrometer were set as follows: ESI voltage: +3 kV; in-source CID: 15 eV; S-lens RF level: 80%; capillary temperature: 360 °C. To facilitate the analysis of the whole slide area, we use a Velos ion transfer tube with 2 cm extension (Scientific Instrument Services Inc.).

### High Contrast Microscopy

Whole slide images were taken using LionHeart Automated Microscope BioTek microscope (Agilent Technologies, Santa Clara, CA). A 4× objective was used for fast acquisition with a 13% overlap between individual images allowing proper image stitching and formation of a single view for entire glass slide. Images were processed and exported as .tiff files. The number of cells on slide was manually determined using point selection function in QuPath,²⁴ which allowed for the export of the spatial coordinates of the cells as a .csv file. Downstream throughput calculations were performed using a MATLAB script developed in-house.

scPiMS Data Acquisition

scPiMS data acquisition was performed in the individual ion mass spectrometry (I²MS) mode on a previously described Orbitrap Q-Exactive Plus mass spectrometer (Thermo Fisher Scientific GmbH, Bremen, Germany).¹⁶ The spectral acquisition rate was set at one scan per second corresponding to a mass resolution of 70,000 at m/z=200. During scPiMS data acquisition, proteoforms from single cells were sampled and ionized by a nano-DESI probe to generate multiply-charged ions distributed across multiple charge states. Ions in one detection period were collected in the individual ion regime, which gives a singular ion signal at a defined m/z (or frequency) value. Due to the protein signals emerging from single cells, the majority of the scPiMS ion detection periods were dominated by individual ion signals even at extended MS injection times over 200 milliseconds. The Orbitrap central electrode voltage was adjusted to −1 kV to improve the ion survival rate in I²MS.²⁵ Injection time and HCD gas pressure level for scPiMS were systematically optimized in this study. Details in optimizing MS parameters (injection time and HCD gas pressure setting) are discussed in the “scPiMS Optimization” section. In the high-throughput experiments, an injection time of 700 milliseconds and an HCD gas pressure setting of 0.5 (High Vacuum 2×10⁻⁵ Torr; UHV pressure <5×10⁻¹¹ Torr) was used to reduce collision-induced ion decay within the Orbitrap analyzer without sacrificing the trapping efficiency for intact protein ions.²⁵

Additional relevant data acquisition parameters were as follows: mass range: 500-2500 n z; AGC mode: disabled; enhanced Fourier transform: off; averaging: 0; microscans: 1. Time-domain data files were acquired at detected ion frequencies and recorded as Selective Temporal Overview of Resonant Ions (STORI) files;²⁶ STORI setting: Enabled.

scPiMS Feature Selection & Data Analysis

Analysis of scPiMS data was performed using MATLAB and a C^{#} script developed in-house. scPiMS raw data were collected as continuous chronograms, dubbed as “cellograms” comprised of discrete cell extraction profiles and other peak features from chemical noise on the glass slide.

In high-throughput raster scanning mode, we employed a raster scan rate of 30 μm/s and a probe size of ~250 μm. The ion chronogram containing processed individual ions was employed for peak picking with an ion count threshold of 500 and a minimum peak distance of 350 m. The peak features picked by the program were further categorized into single-cell or multi-cell features in the cellogram using characteristics of the peaks. In particular, if the half-width-at-half-maximum on the right side of a cellogram was more than 250 μm, the feature was recognized as a multi-cell feature. Multi-cell features were aggressively filtered in this step and only the single cell features were used for further analysis.

The STORI files of the MS scans from the single cell features were extracted from the dataset and concatenated to construct a single mass-domain spectrum. In particular, all individual ion signals were resubjected to charge state assignment for mass calculation. The neutral masses of the protein ions were calculated by the well-known Equation 6:

$$Mass = ( \frac{m}{z} \times z ) - ( z + {M}_{proton} ) \qquad ( 6 )$$

Charge state (z) is obtained from the slope of induced image current determined by the STORI analysis described in detail elsewhere.²⁶ Accurate charge assignment of each ion was statistically evaluated by comparing the slopes of its isotopologues across different charge states from the entire dataset.

### Proteoform Identification Via Intact Mass Tags (IMTs)

The summed mass-domain ion library was converted to .mzML format and processed using a custom version of TD Validator (Proteinaceous, Evanston, IL) implemented with an MS¹ IMT search function. The sum of mass spectra from targeted cells was self-calibrated according to the accurate masses of three identified proteoforms in the 10-60 kDa mass range. A custom proteoform database constructed from SwissProt of Mus musculus was used for searching. The IMT search was performed with a ±1.5 part-per-million (ppm) mass tolerance considering Methionine on/off, monoacetylation, and monophosphorylation as possible proteoform modifications in the database (i.e., a search space of ~60000 proteoforms). Additional proteoform matches were curated by spectral inspection and manual annotation of putative modifications. The IMT annotations were further employed for mass calibration of single ions involved in the same I²MS data processing to account for mass calibration differences of the instrument on different days.

Targeted scPiMS of Single Cells

A suspension of NIH 3T3 cells were drop cast onto a glass slide covalently functionalized with poly-lysine-like moieties. The modified surface of the slide facilitates cell adhesion to the glass slide for downstream scPiMS analysis. To reduce the matrix effect from the high-salt buffer residue and enhance protein detection, we developed an in situ alcohol fixation and wash protocol, which fixed the protein content in surface-adhered single cells without disturbing their spatial locations. A serum-free incubation buffer (1× phosphate buffer saline, 1×PBS) was employed in the workflow as we observed severe cell dislocation on the surface when DMEM with 10% FBS was used as the cellular incubation buffer.

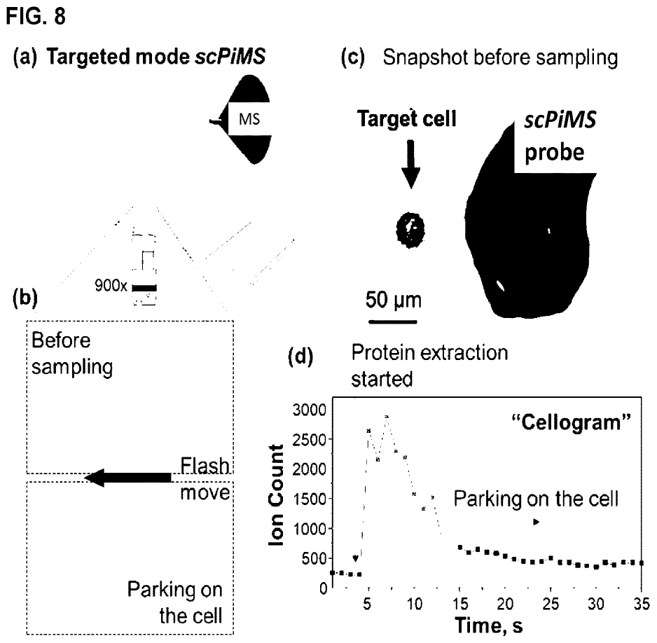

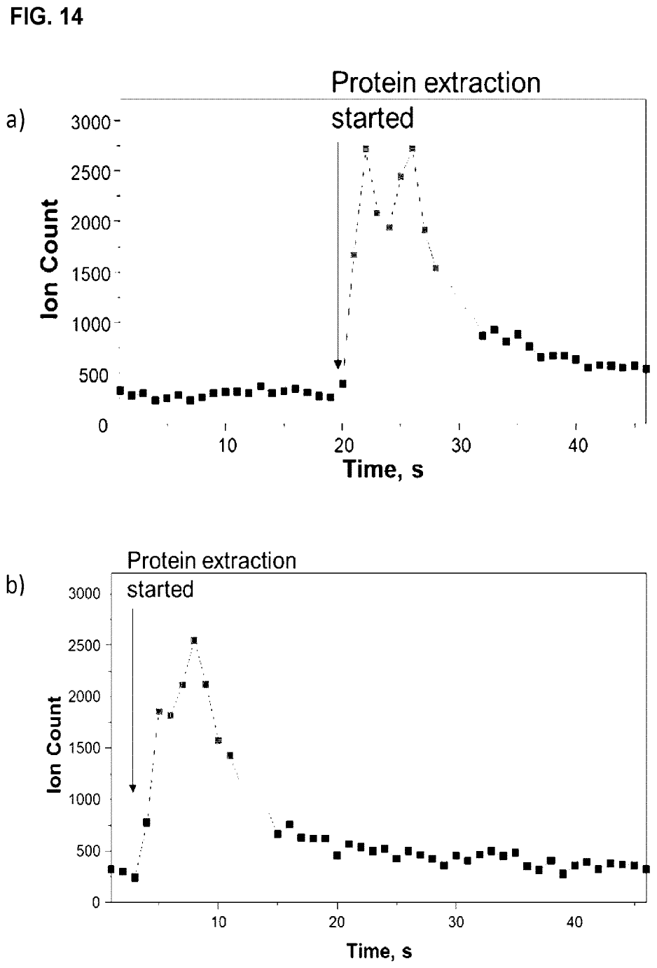

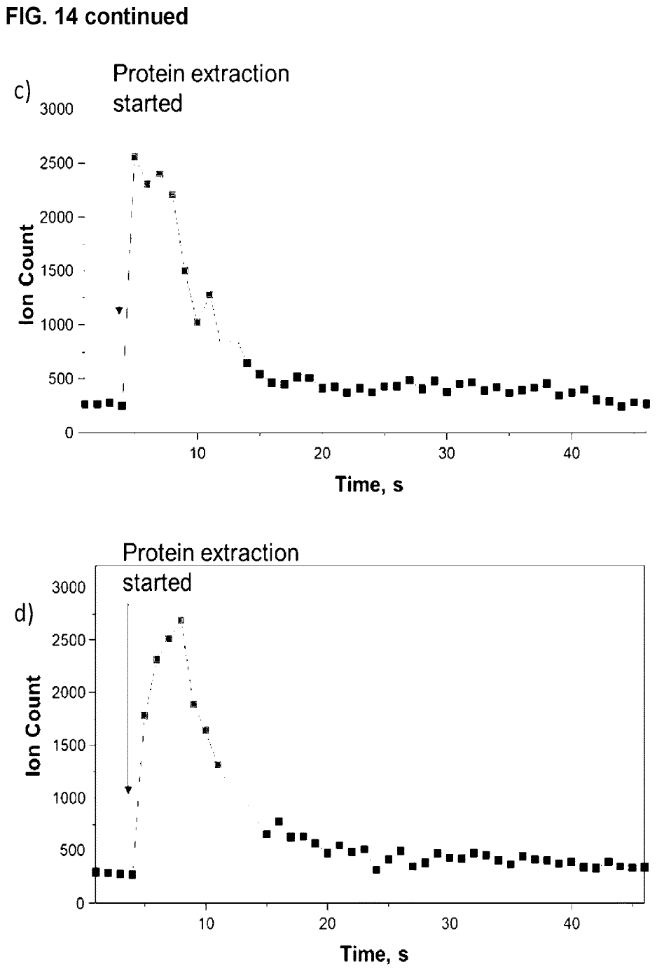

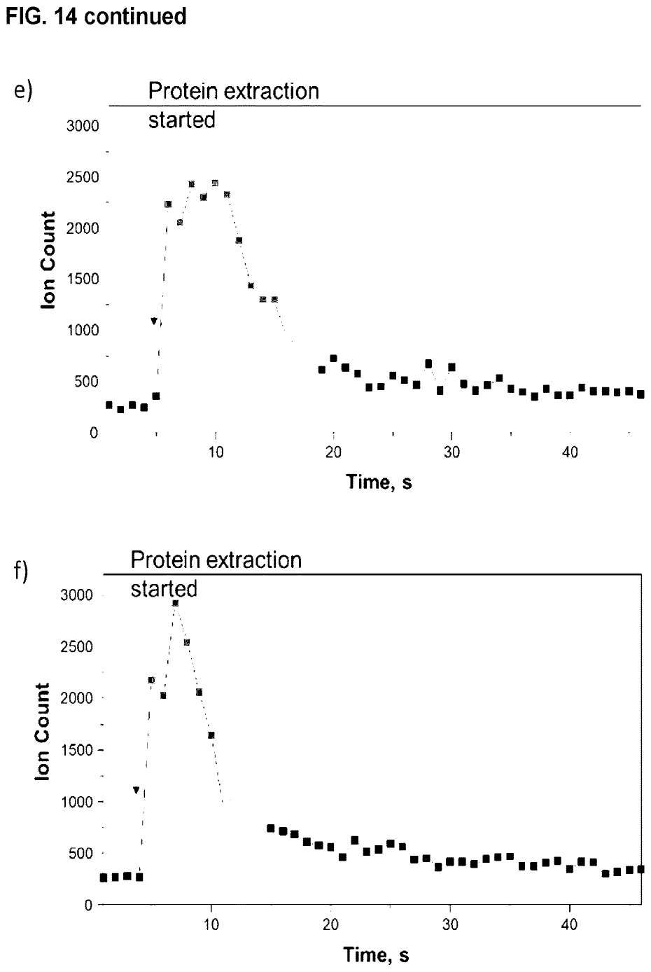

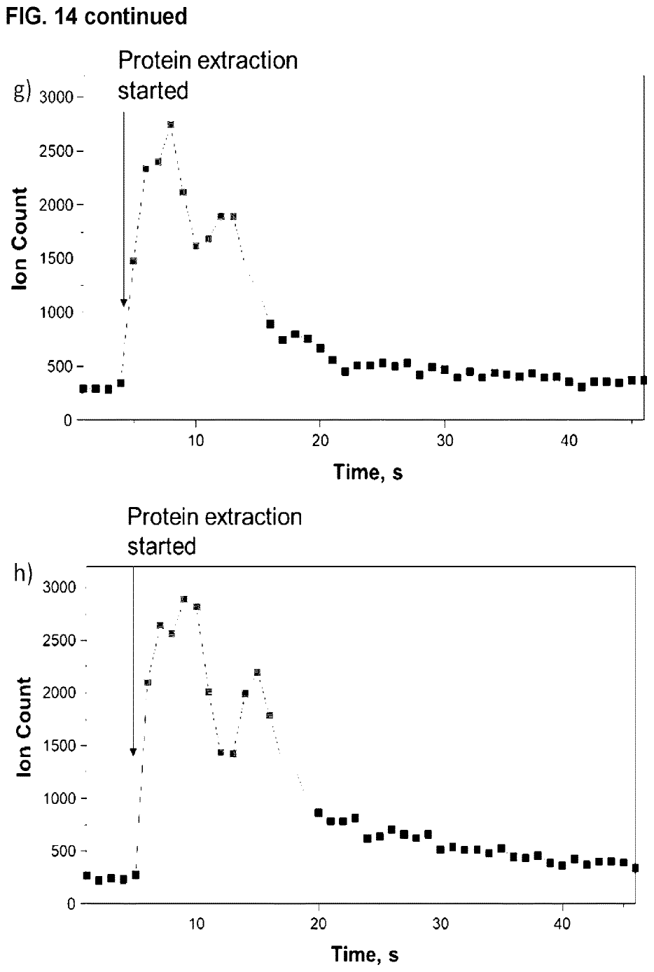

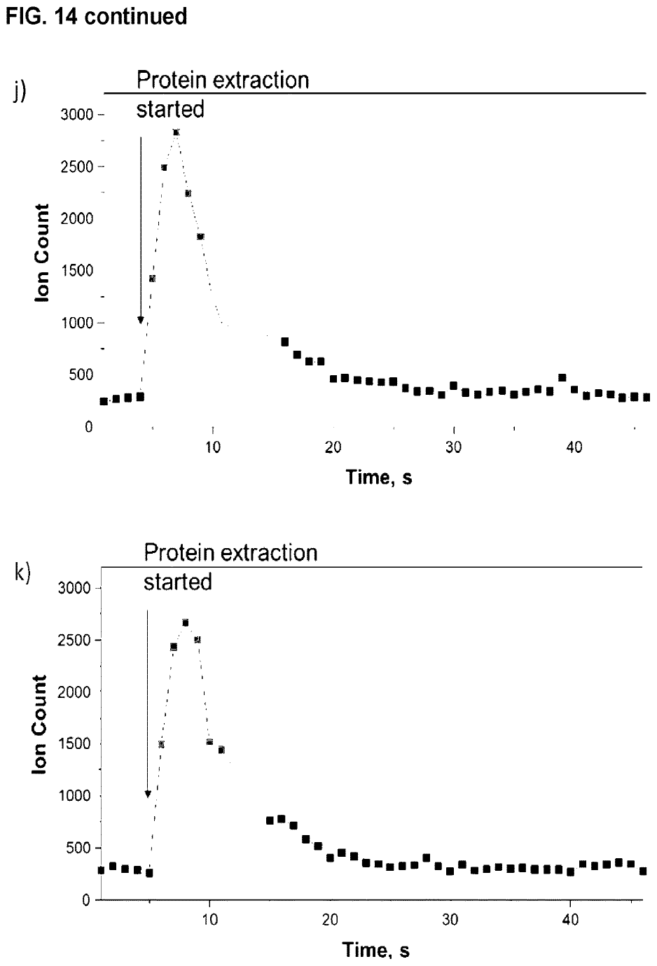

We first applied scPiMS to targeted single cells with the aid of a high-magnification microscope under the glass slide (FIG. 8a, “targeted mode”). The 900× high-magnification microscope allowed for the visualization of a fixed single cell and the scPiMS extraction process. In targeted mode, a single cell feature was first identified by the 900× microscope while the nano-DESI probe stayed ~100 μm above the slide without making a liquid contact. Once a cell of interest was identified, the nano-DESI probe was brought into contact with the glass surface at a location ~100 μm away from the single cell to activate surface sampling. Following adjustment of the microscope to bring the cell in focus, the cell was brought into the liquid bridge with manually controlled stage motions to initiate MS data acquisition by I²MS (FIG. 8b). FIG. 8c shows a snapshot of the selected targeted cell right before scPiMS sampling was triggered. In the total individual ion count chronogram shown in FIG. 8d, we observed a peak corresponding to protein ion detection from the single cell over time. In particular, the total ion count quickly reached its apex just one second after the initial signal started to appear, and the main burst of proteoform signals resembled a chromatographic peak (FIG. 8d). The peak contained 8 scans for 8 seconds, with ion counts above full width at half maximum (FWHM, red dots in FIG. 8d). Further extraction while parking on the cell did not result in more scans with ion count significantly higher than the baseline prior to protein extraction on the cell. We note that more protein signals may be observed for the same single cell if the scPiMS solvent condition is changed, and the morphology of the cells was largely retained post scPiMS analysis. Similar chronographic protein extraction features were obtained on additional ten single cells on slide in this targeted, manual mode with reproducible FWHM and peak shapes (FIG. 14). This peak shape is consistent with extraction kinetics of cellular protein, and led to referencing a time-resolved collection of such peaks as “cellogram” (vide infra).

Optimization of scPiMS

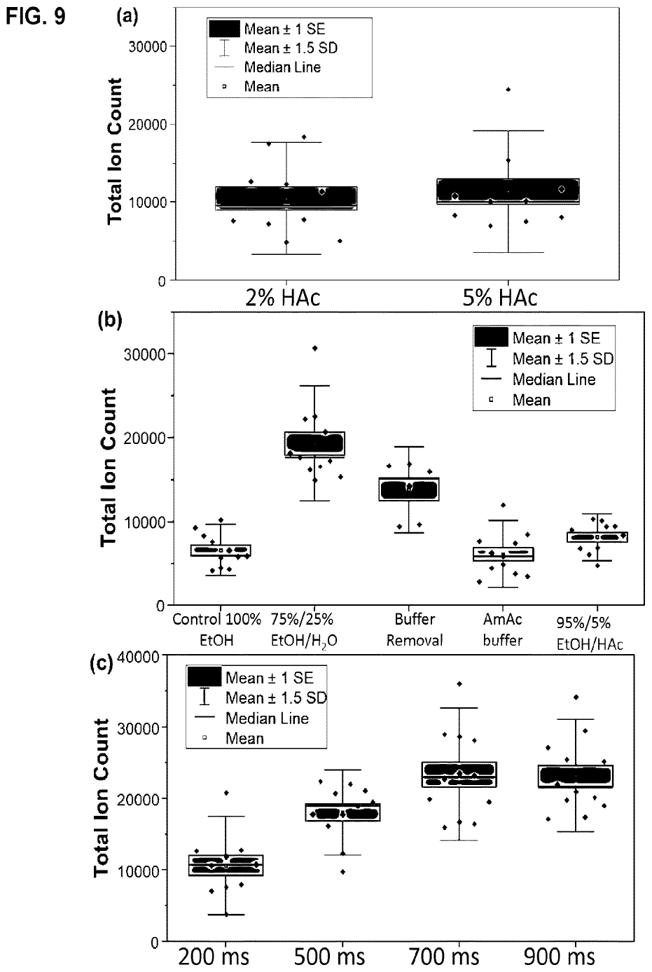

We next sought to understand the factors that affect protein extraction and ionization efficiency in scPiMS for maximizing single cell proteoform detection. We used the total individual ion count detected per cell within the cellogram as an initial metric while the same MS settings were used (injection time=500 ms, HCD pressure setting=0.5). To compare different cells and conditions, we included the ion counts from the scans within FWHM and those that are 3 times higher than the background prior to protein extraction (light dots in FIG. 8d). All of the ions considered in the total ion count were subjected to I²MS data processing and were validated as multiply-charged individual ions. The total ion counts for cells analyzed in different conditions are summarized in FIG. 9a. Approximately 10 single cell experiments were performed in each condition and the results tabulated.

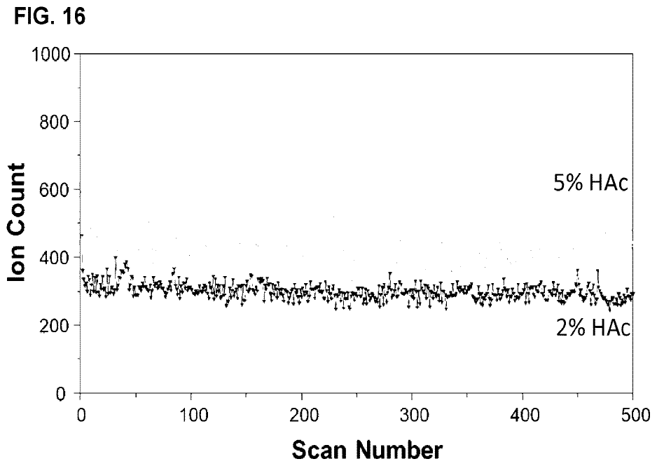

We first examined the effect of nano-DESI extraction/ionization solvent conditions, which have been shown to enhance bias in the detection of different classes of biomolecules.²⁷ In particular, we compared three levels of acetic acid (0.6%, 2%, and 5% in 60%/40% acetonitrile/water) using the 100% cold ethanol fixation protocol. Using 2% or 5% acetic acid allowed for the detection of an average of >5-fold higher number of ions per cell compared to 0.6% acetic acid (FIG. 9a and FIG. 15). Despite the similar single cell ion count obtained in both conditions, we selected 2% acetic acid for the following scPiMS experiments due to lower background noise level while scanning on a blank area of the slide (FIG. 16).

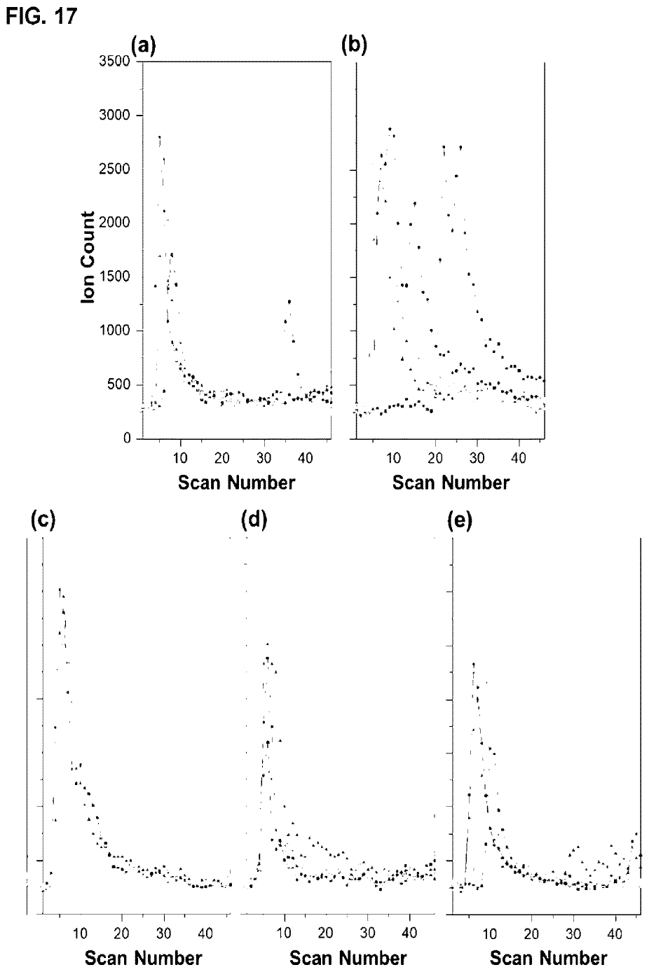

Protein extraction is affected by both fixation in cell preparation step and on-the-fly liquid extraction by nano-DESI. Moreover, the presence of salt in the PBS incubation buffer may negatively affect the electrospray ionization efficiency of proteins. We summarized the total ion count in different fixing conditions in FIG. 9a (also see cellograms in FIG. 17). The steps in the five workflows tested for alcohol fixation are summarized in FIG. 18. All slides were rinsed with 75%/25% ethanol/water as a final step to obtain a similar chemical background in all tested conditions. Compared to the adding cold 100% ethanol directly into the chamber slide containing PBS buffer and cells, we obtained significantly more total ion detection from single cells when 100% ethanol was replaced by cold 75%/25% ethanol/water mixture as the fixing solution (FIG. 9b). Removal of PBS buffer from the chamber prior to the addition of fixing solution also facilitated total ion detection, however, we observed under the 900× microscope that more cells were mechanically ruptured or lysed, which may be attributed to the drying step before adding the fixing solution (an example of ruptured cells FIG. 18c). Moreover, we examined ammonium acetate as an MS-compatible buffer instead of PBS as the solution environment during in situ fixation, which yielded similar number of total ions as the control condition. Furthermore, 5% acetic acid in ethanol has been shown to facilitate solvent penetration into cells and enhance in situ protein fixation, which only showed a moderate total ion count improvement over 100% ethanol and was not comparable to the optimal fixation condition (FIG. 9b). The appearance of the cells under these different fixing conditions were shown in FIG. 18. Based on the above experiments, we concluded that cold 75%/25% ethanol/water mixture gave the optimal scPiMS performance for in situ fixation.

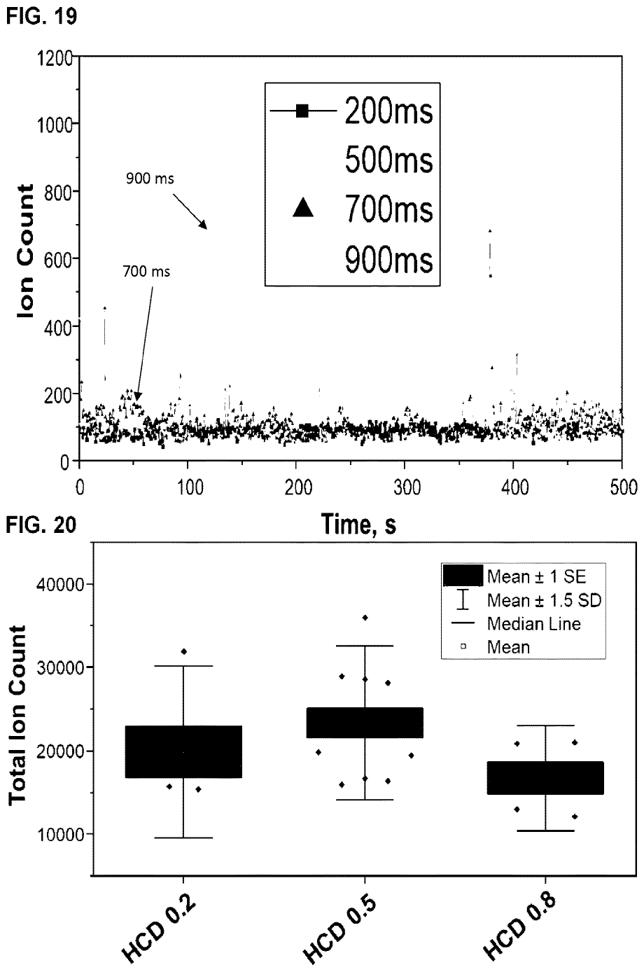

We further optimized the MS settings for the detection of more ions per Orbitrap detection period (FIG. 9c). A higher number of ions may be captured by optimizing the settings affecting the ion collection step in the C-trap. In particular, injection time determines the number of ions allowed into the C-trap per acquisition period, while the trapping gas pressure plays an important role in collisional cooling and efficient capturing of the ion cloud. FIG. 9c shows a summary of the total ion collection of ~10 targeted cells and background ion counts at different injection times. When pressure setting of 0.5 was employed, total number of ions per cell and background ion count increased as a function of injection time up to 700 ms. Further increase in injection time to 900 ms did not result in higher total ion count. Moreover, 900 ms resulted in significantly higher background ion count compared to injection times equal or less than 700 ms (FIG. 19). As a result, we selected 700 ms as the optimal injection time for scPiMS data acquisition. We note that the background ion count may be affected by the instrument background noise level and chemical noise from the ion source, which should be evaluated prior to data acquisition. For HCD gas pressure setting, more ions were detected at pressure setting of 0.5 compared to 0.2 and 0.8, however, the increase was not significant (FIG. 20). Our previous studies have discussed the effect of HCD gas pressure on the ion collection efficiency for proteins of different masses, and this will be discussed for scPiMS in the next section.

Proteoform Assignment Score (PAS) for scProteoforms

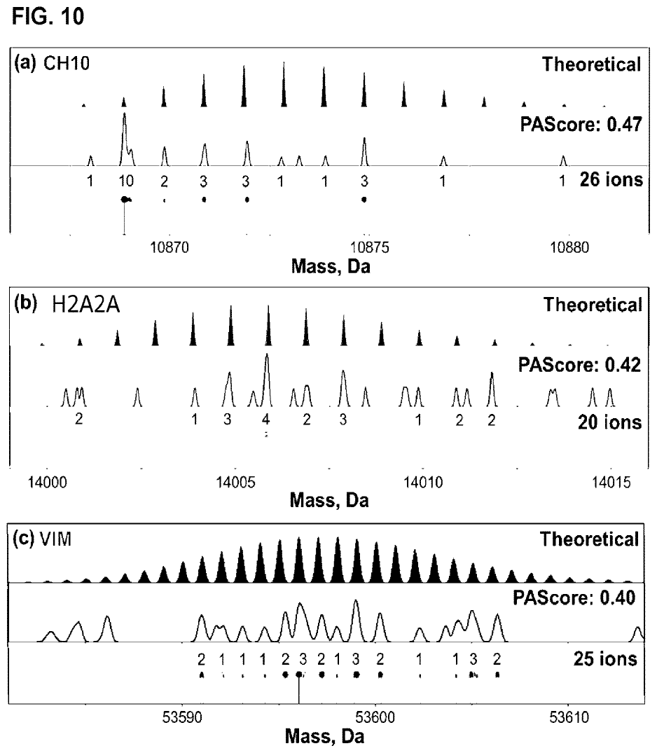

Discrete ion counts assigned to isotopic distributions for specific proteoform masses is a new challenge encountered when using single ion detection from single cells. To accomplish charge, mass and proteoform assignment in this new regime, we first aggregated the data from ~50 single cells to construct a total mass-domain spectrum. Using this ‘aggregated’ mass spectrum, we detected isotopic distributions for the identification of proteoform masses using the THRASH algorithm²⁸ and then query a database of proteoforms using intact mass tag (IMT) approach.¹⁹ In FIGS. 10a-10c, we show three scProteoform cases where 20, 25 and 26 ions were matched with isotope masses within 0.3 Da for proteoforms of N-acetylated 10 kDa heat shock protein (CH10), histone H2A type 2-A (H2A2A), and N-acetylated vimentin (VIM), a well-known filament protein expressed in NIH 3T3 mouse fibroblast cells.²⁹

Similar to prior tissue imaging experiments for proteoforms,¹⁹ we used the total scPiMS dataset for the extraction of the mass spectrum of each single cell. To process this single-cell proteoform data, we developed an informatic framework to assign proteoforms in single cells. This framework was tailored to cases where isotopic distributions were both well populated, and those that were populated only sparingly. Given a set of ion masses that align with the isotopologues of known or candidate proteoform, a new score could assert the confidence of its detection in the collection of 10-20 k proteoform ions detected in a single cell. To capture the confidence of these single-cell proteoform detection events (scProteoform) based on the combined presence of matched isotope ions, we developed a Proteoform Assignment Score (PAScore). The PAScore for these 3 cases were 0.47, 0.40 and 0.42, respectively, results explained in further detail and context below.

The PAScore is based on a mathematical model which considers both the mass accuracy and relative abundance of the isotopologue masses of the ions (Eq. 7). In matching a group of individual ions to a reference proteoform using the PAScore system, each ion was first matched to its isotopologue with a mass tolerance of 0.3 Da. The probabilistic score was calculated based on the inclusion-exclusion principle by Eq. 7:

$$PAS = 1 - {\prod }_{k = 1}^{n} [ 1 - ( {P}_{isotopologue} \times {P}_{mass error} ) ] \qquad ( 7 )$$

where P_{isotopologue} is the expected relative intensity of the matching isotopologue, and P_{mass error} is a probability score for the mass error-based between the observed individual ion mass and the theoretical mass of the isotopologue. The PAScore utilizes the cumulative distribution function (CDF) of a normal distribution with a mean and standard deviation determined by the theoretical mass and width of an isotopic peak, respectively:

$${P}_{mass error} = \{ \begin{matrix}2 * CDF ( {m}_{ion} ) & {m}_{ion} < {m}_{iso} \\ 2 * [ 1 - CDF ( {m}_{ion} ) ] & {m}_{ion} > {m}_{iso}\end{matrix} \qquad ( 8 )$$

where mᵢₒₙ is the mass of the individual ion and mᵢₛₒ is the theoretical mass of the matched isotopologue.
PAS-Based scPiMS Optimization Using scProteoforms

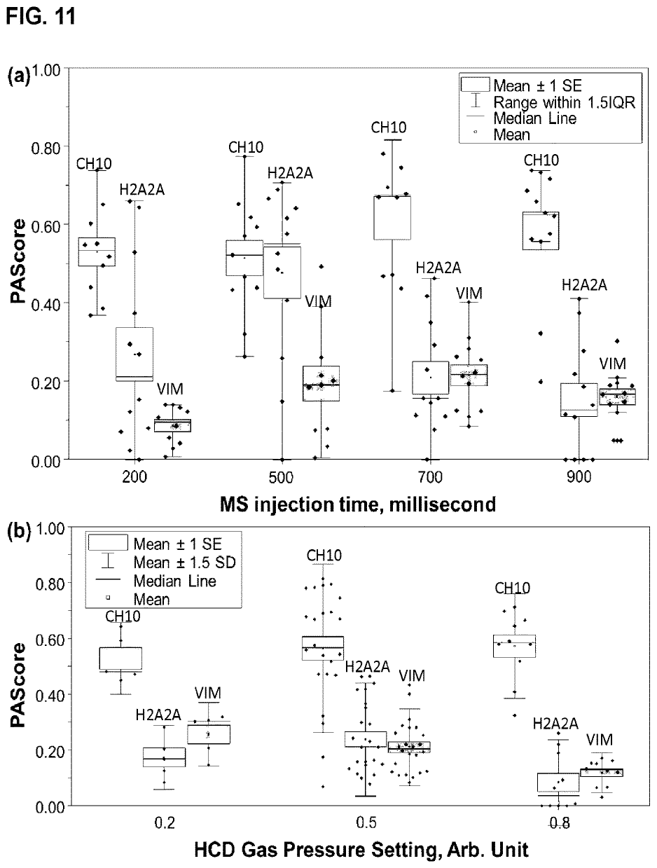

Using the procedure described above, we were able to optimize this new method using identified scProteoforms. We selected a few proteoforms that were MS¹-annotated with high confidence and detected over abroad mass range, including CH10 (10.8 kDa), H2A2A (14.2 kDa), and VIM (53.5 kDa). These proteoforms were previously characterized in mouse tissues by PiMS and tandem MS enabled by the AutoPiMS workflow.^{19, 20} FIG. 11a shows the PAScores of the three proteoforms at different injection times. Despite the PASs of CH10 remaining relatively constant across all the injection times tested, the PAS of H2A2A showed an increase from 200 ms to 500 ms and decreased by over 2-fold at higher injection times. In contrast, PAS of VIM showed an increase from 200 ms to 500 ms and further increased at 700 ms, then decreased at 900 ms injection time. These observed PAS trends may be explained by the space charge directed ion dynamics in the C-trap at long trapping periods. In particular, introducing more ions over time into the C-trap results in a higher total space charge, which repels ions out from the ion cloud at the center of the trap. In this process, ions of low mass and high charge states acquire higher velocity or more kinetic energy to escape the trap and suffer from higher rate of losses.³⁰ This is well evidenced by the significant loss of lower-mass H2A2A (~14 kDa) and increase in VIM (~54 kDa) between 500 ms and 700 ms injection time. To better capture the more components of the proteome particularly the high mass proteoforms, we sought to use an injection time of 500 ms to 700 ms to reduce the mass-related biases. This also takes both total ion count per cell and background noise level into consideration as discussed in previous sections.

FIG. 11b shows the PASs for the three proteoforms at different HCD gas pressure settings. In previous work, we have characterized the HCD gas pressure dependence of I²MS performance for proteoforms of different masses.³¹ Briefly, higher pressure gives rise to more efficient collisional cooling in the C-trap, while it negatively affects the lifetime of individual ions in the Orbitrap particularly for large denatured proteoforms. The trend of PAS as a function of pressure is consistent with our previous findings. In particular, we observed a slight increase in PAS of CH10 and H2A2A from pressure setting at 0.2 to 0.5, indicating more efficient ion collection at higher pressures. The scProteoforms of H2A2A were not well detected at pressure setting of 0.8. In contrast, PASs of VIM showed a negative correlation with pressure, consistent the finding that more significant ion losses are expected for high mass denatured proteoforms at higher pressures due to higher probabilities of ion decay induced by energetic collisions in the ion detection period. For optimal ion survival and single cell proteoform detection, the HCD gas pressure setting of 0.5 was selected for all subsequent scPiMS experiments.

scPiMS at Higher Throughput

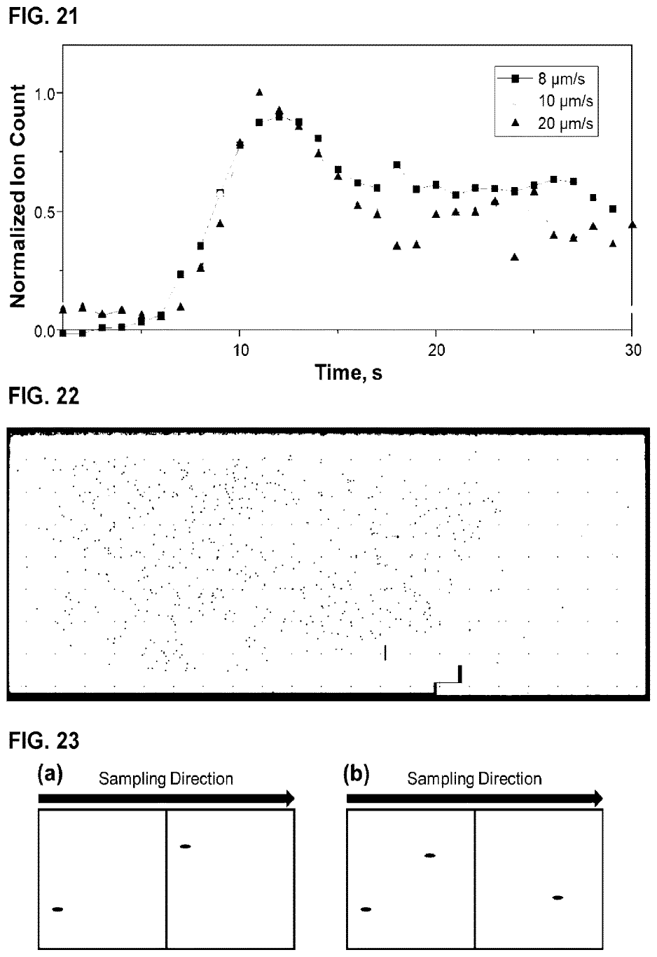

Almost complete sampling of single cells within a few seconds provides a unique opportunity to use raster scanning for scPiMS with the potential to scale the sampling of single cells. Therefore, raster scanning was used to sample cells on a slide with a nano-DESI probe move in parallel lines at a constant linear velocity.¹⁹ The spacing between adjacent lines equals the probe size, which ensured all the surface area of the slide containing single cells was covered. To compare scPiMS protein extraction efficiency in the raster scanning mode versus targeted mode, we used a nano-DESI probe with a 200 μm footprint size to scan across a few single NIH 3T3 single cells. To ensure enough sampling time in the raster scanning mode, we tested the scan rate of the probe at 8 μm/s, 10 μm/s, and 20 μm/s, which correspond to an extraction time of 25 s, 20 s, and 10 s, respectively, during which the liquid bridge was in contact with the cell for extraction. The cellograms obtained at three different scan rates show similar decay profiles (FIG. 21), which demonstrates that the protein extraction kinetics were not affected by the motion of the probe as long as the liquid bridge stayed in contact with the cell region. The average number of ions obtained are comparable with targeted cell experiments, indicating that the raster scanning mode is equally efficient in single cell sampling. This allowed us to design the following high throughput experiments, in which the scan rate was set such that the liquid bridge leaves a cell by the time the full cellogram is captured. The scan rate may be calculated using Eq. 9:

$$Raster scanning rate = \frac{Probe size + single cell size}{Exposure time} ≅ \frac{Probe size}{Exposure time} \qquad ( Eq . 9 )$$

where exposure time equals the total length of the single ion cellogram (~10 s for NIH 3T3 cells as demonstrated in the targeted experiments). When the size of the single cells is negligible compared to the probe size, the probe size may be used to directly calculate the proper raster scanning rate.

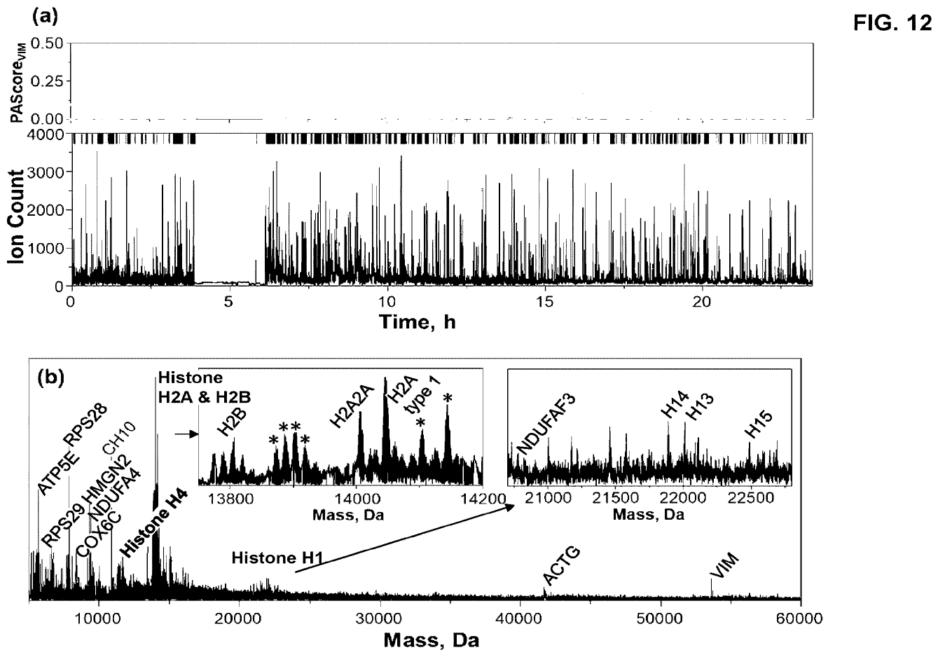

We next performed scPiMS experiment in the raster scanning mode on a whole slide, containing ~538 single cells (FIG. 22). The experiment lasted for ~20 hours continuously and in an unattended fashion. The spatial coordinates of the cells were manually annotated against a high-contrast optical microscopy image (FIG. 22). We employed a raster scan rate of 30 μm/s and a probe size of ~250 μm. Using the algorithm to extract single cell features from the entire cellogram excluding 2-cell or multi-cell detection events, we found a total of 461 protein extraction events that showed the characteristics of single cell detection (FIG. 12, lower panel). Calculation of a theoretical number of single cell detection events according to the spatial coordinates of optically-registered single cells gave 514 (Experimental Section), corresponding to a ~90% experimental success rate in single cell detection. The average number of ions detected per cell is 7200 with a variance of 5200 ions at 1 sigma.

Next, the MS scans from the 461 single cell features in the cellogram were aggregated and subjected to I²MS processing to generate a total mass spectrum of NIH 3T3 cells. In particular, the THRASH algorithm asserted 126 proteoforms up to 60 kDa, and 38 were manually annotated using intact mass against a list of proteoforms from the Mus musculus SwissProt database within a ±1.5 part-per-million (ppm) mass tolerance. Larger proteoforms beyond 60 kDa are challenging to identify due to more isotopic channels, limited solubility in the electrospray solvent, and in some cases higher molecular heterogeneity due to multiple post-translational modifications. Aside from the three proteoforms discussed earlier (CH10, H2A2A, VIM), the aggregated data captured the familiar landscape of core and linker histones (H1, H2A, H2B, H4, FIG. 12b, inset). Other identified proteoforms include those of abundant proteins (ACTG), high-mobility group proteins (HMGN1, HMGN2, HMGN3), ribosomal proteins and subunits of the electron transport chain of oxidative phosphorylation (FIG. 12b). The scatter plot at the top of FIG. 12a shows the PAScores of scProteoforms of monoacetylated vimentin (VIM) in the 461 single cells. Of the 461 cell detection events in the cellogram, 155 showed detection of evidence of VIM with non-zero PAScores.

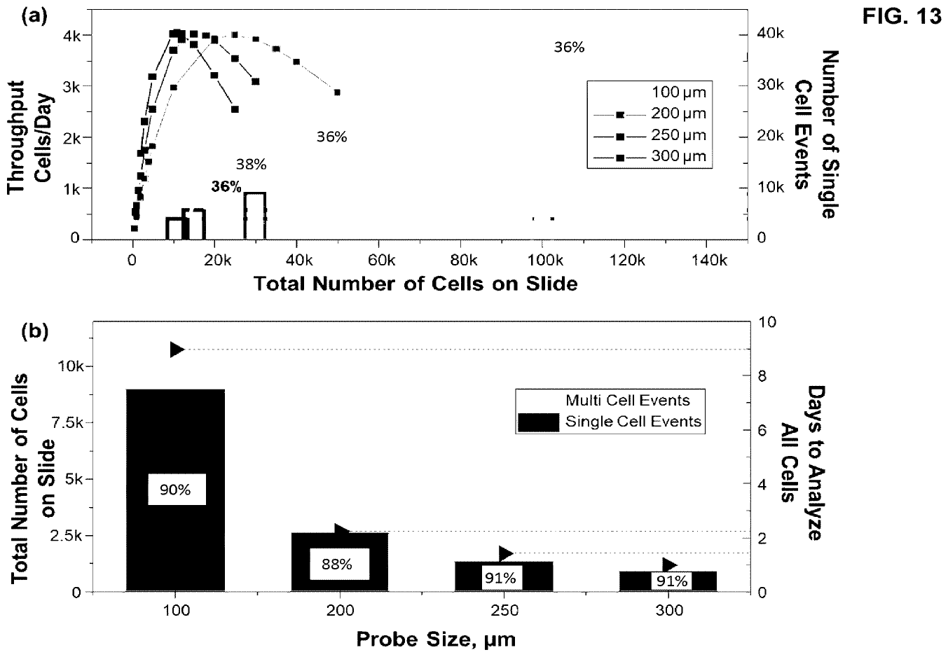

To understand the potential of scPiMS for the higher throughput, we carried out statistical modeling using different probe sizes (100 μm to 300 μm) and cell densities (number of dropcasted cells over a fixed surface area), where single cell coordinates were randomly generated in the slide area at different number densities. The method for estimation of theoretical throughput of scPiMS is described in Supplemental Information and FIG. 23. Briefly, the modeling utilized a Monte Carlo method that generates 1000 random cell distributions at different number densities, from which throughput together with true single cell events vs. multiple cell events were determined with statistical significance. An upper bound for scPiMS throughput in the configuration presented here is ~4000 cells per day independent of the probe size (FIG. 13a). However, this throughput would come with a cost, as <40% of cells would be detected alone and the protein profiles from the rest would be obscured due to sampling overlap with surrounding cells (FIG. 13a). Further modeling suggested that a theoretical throughput of ~1000 cells per day allows for ~90% of the cells being analyzed as singles (FIG. 13b). This requires the combination of the suitable cell density for different probe size shown in FIG. 13b. In practice as a standard setting, using a CC²-functionalized glass slide (45 mm×15 mm) containing ~2000 randomly adsorbed cells, we could perform scPiMS with a probe size of ~200 μm at a raster scanning rate of 30 μm/s and a line spacing of 200 μm. This setup would allow for a throughput of ~1000 cells per day with 90% of the total cell population analyzed as single cells.

In conclusion, we establish a new approach to single-cell proteomics with the potential of high-throughput cell type classification using a simple liquid-based MS imaging probe without antibodies or labels. This work translates the potential of single ion technology into a scalable approach to single-cell proteomics for fast molecular profiling and identification with proteoform specificity up to ~60 kDa. We demonstrate a raw single cell analysis rate of <10 seconds per cell, and project the feasibility of processing ~1000 single cells per day, a several fold increase compared to the current single cell proteomics techniques using bottom-up proteomics.^{10, 32, 33}

The scPiMS platform also converts the ‘no-digestion’ or ‘top-down’ approach to proteomics into a key advantage that could potentially help in widening an important bottleneck in SCPs, yet involves interpretation of a new data type. To our knowledge, no score currently exists for how effective these scProteoform ions can be assigned making this the first report of such a metric like the PAScore. We acknowledge that using scProteoform assignment of proteoforms using a group of single ions might be unreliable for low abundance proteins in single cells. However, as the key ‘unit of measurement’ and a new concept for the SCP community, our future work will focus on the reproducibility and proteome coverage of scPiMS advancing our understanding to major sources of bias and quantitative performance. The scPiMS platform will be advantaged using a reference set of known proteoforms and their ions to both accelerate the proteoform assignment step and increase the depth the analysis possible to obtain in each cell. By providing proteoform-level information, scPiMS has the potential to characterize a large number of single cells of mixed cell types from blood or those disaggregated from tissues. To the extent proteoform measurement connects to complex phenotypes, an advanced version of scPiMS may create opportunities to advance single cell biology provided it can be improved in the future.

scPiMS Throughput Calculation

The ultimate throughput is determined by the number of single cells analyzed in unit time calculated by Eq. 10:

$$Throughput = \frac{Number of true single cell events}{Total scan time over the slide} = \frac{Number of true single cell events}{\frac{{y}_{total}}{probe size} \times \frac{{x}_{total}}{raster scanning rate}} \qquad ( Eq . 10 )$$

where xₜₒₜₐₗ and yₜₒₜₐₗ are the dimensions of the entire slide area with cells, and raster scanning rate is determined by the probe size (Eq. 9). The highest theoretical throughput may be achieved using a proper probe size to sample cells of a particular number density.

A true single cell event is characterized by a cellogram that does not overlap with its adjacent cellograms in a high-throughput raster scanning experiment. For a certain probe size, a favorable raster scanning rate is selected so that the whole cellogram corresponding to complete cell sampling is captured, which is described in Eq. 3. With the combination of probe size and scan rate, we can define a “single cell grid” corresponding to an area where a single cell event may be expected, if only one cell is present in the grid (FIG. 23a). The dimensions of the squared-shaped grid equal the probe size. Multiple cell events will take place if more than one cells are located in the same grid as the cellogram has not fully waned before the probe runs into the next cell in the same grid (FIG. 23b). We note that the grid approach only defines the upper bound of single cell events. The practical number of true single cell events may be lower due to the Poisson-distributed cell loading process, in which each assumed single cell event needs to be validated. The validation may be performed by calculating the distance of the single cell with cells in its neighbor grids.

### REFERENCES

(1) Melani, R. D.; Gerbasi, V. R.; Anderson, L. C.; Sikora, J. W.; Toby, T. K.; Hutton, J. E.; Butcher, D. S.; Negrão, F.; Seckler, H. S.; Srzentić, K.; et al. The Blood Proteoform Atlas: A reference map of proteoforms in human hematopoietic cells. Science 2022, 375 (6579), 411-418. DOI: 10.1126/science.aaz5284 (accessed 2023/05/08).
(2) Smith, L. M.; Kelleher, N. L. Proteoforms as the next proteomics currency. Science 2018, 359 (6380), 1106-1107. DOI: 10.1126/science.aat1884 (accessed 2023/05/08).
(3) Gawad, C.; Koh, W.; Quake, S. R. Single-cell genome sequencing: current state of the science. Nature Reviews Genetics 2016, 17 (3), 175-188. DOI: 10.1038/nrg.2015.16.
(4) Burnum-Johnson, K. E.; Conrads, T. P.; Drake, R. R.; Herr, A. E.; Iyengar, R.; Kelly, R. T.; Lundberg, E.; MacCoss, M. J.; Naba, A.; Nolan, G. P.; et al. New Views of Old Proteins: Clarifying the Enigmatic Proteome. Mol. Cell. Proteomics 2022, 21 (7), 100254. DOI: https://doi.org/10.1016/j.mcpro.2022.100254.
(5) Mund, A.; Coscia, F.; Kriston, A.; Hollandi, R.; Kovacs, F.; Brunner, A.-D.; Migh, E.; Schweizer, L.; Santos, A.; Bzorek, M.; et al. Deep Visual Proteomics defines single-cell identity and heterogeneity. Nat. Biotechnol. 2022, 40 (8), 1231-1240. DOI: 10.1038/s41587-022-01302-5.
(6) Dou, M.; Clair, G.; Tsai, C.-F.; Xu, K.; Chrisler, W. B.; Sontag, R. L.; Zhao, R.; Moore, R. J.; Liu, T.; Pasa-Tolic, L.; et al. High-Throughput Single Cell Proteomics Enabled by Multiplex Isobaric Labeling in a Nanodroplet Sample Preparation Platform. Anal. Chem. 2019, 91 (20), 13119-13127. DOI: 10.1021/acs.analchem.9b03349.
(7) Budnik, B.; Levy, E.; Harmange, G.; Slavov, N. SCoPE-MS: mass spectrometry of single mammalian cells quantifies proteome heterogeneity during cell differentiation. Genome Biol. 2018, 19 (1), 161. DOI: 10.1186/s13059-018-1547-5.
(8) Petrosius, V.; Aragon-Fernandez, P.; Uresin, N.; Kovacs, G.; Phlairaharn, T.; Furtwangler, B.; Op De Beeck, J.; Skovbakke, S. L.; Goletz, S.; Thomsen, S. F.; et al. Exploration of cell state heterogeneity using single-cell proteomics through sensitivity-tailored data-independent acquisition. Nat. Commun. 2023, 14 (1), 5910. DOI: 10.1038/s41467-023-41602-1.
(9) Matzinger, M.; Mayer, R. L.; Mechtler, K. Label-free single cell proteomics utilizing ultrafast LC and MS instrumentation: A valuable complementary technique to multiplexing. Proteomics 2023, n/a (n/a), 2200162, https://doi.org/10.1002/pmic.202200162. DOI: https://doi.org/10.1002/pmic.202200162 (accessed 2023/05/08).
(10) Ctortecka, C.; Hartlmayr, D.; Seth, A.; Mendjan, S.; Tourniaire, G.; Udeshi, N. D.; Carr, S. A.; Mechtler, K. An Automated Nanowell-Array Workflow for Quantitative Multiplexed Single-Cell Proteomics Sample Preparation at High Sensitivity. Mol. Cell. Proteomics 2023, 22 (12), 100665. DOT: https://doi.org/10.1016/j.mcpro.2023.100665.
(11) Yang, M.; Hu, H.; Su, P.; Thomas, P. M.; Camarillo, J. M.; Greer, J. B.; Early, B. P.; Fellers, R. T.; Kelleher, N. L.; Laskin, J. Proteoform-Selective Imaging of Tissues Using Mass Spectrometry**. Angew. Chem. Int. Ed. 2022, 61 (29), e202200721, https://doi.org/10.1002/anie.202200721. DOI: https://doi.org/10.1002/anie.202200721 (accessed 2022/10/26).
(12) Melby, J. A.; Brown, K. A.; Gregorich, Z. R.; Roberts, D. S.; Chapman, E. A.; Ehlers, L. E.; Gao, Z.; Larson, E. J.; Jin, Y.; Lopez, J. R.; et al. High sensitivity top-down proteomics captures single muscle cell heterogeneity in large proteoforms. Proc. Natl. Acad. Sci. 2023, 120 (19), e2222081120. DOI: 10.1073/pnas.2222081120 (accessed 2023/06/19).
(13) Johnson, K. R.; Gao, Y.; Greguš, M.; Ivanov, A. R. On-capillary Cell Lysis Enables Top-down Proteomic Analysis of Single Mammalian Cells by CE-MS/MS. Anal. Chem. 2022, 94 (41), 14358-14367. DOI: 10.1021/acs.analchem.2c03045.
(14) Yin, R.; Burnum-Johnson, K. E.; Sun, X.; Dey, S. K.; Laskin, J. High spatial resolution imaging of biological tissues using nanospray desorption electrospray ionization mass spectrometry. Nat. Protoc. 2019, 14 (12), 3445-3470. DOI: 10.1038/s41596-019-0237-4.
(15) Laskin, J.; Heath, B. S.; Roach, P. J.; Cazares, L.; Semmes, O. J. Tissue imaging using nanospray desorption electrospray ionization mass spectrometry. Anal. Chem. 2012, 84 (1), 141-148.
(16) Kafader, J. O.; Melani, R. D.; Durbin, K. R.; Ikwuagwu, B.; Early, B. P.; Fellers, R. T.; Beu, S. C.; Zabrouskov, V.; Makarov, A. A.; Maze, J. T. Multiplexed mass spectrometry of individual ions improves measurement of proteoforms and their complexes. Nature methods 2020, 17 (4), 391-394.
(17) Worner, T. P.; Snijder, J.; Bennett, A.; Agbandje-McKenna, M.; Makarov, A. A.; Heck, A. J. Resolving heterogeneous macromolecular assemblies by Orbitrap-based single-particle charge detection mass spectrometry. Nat. Methods 2020, 17 (4), 395-398.
(18) Seeley, E. H.; Caprioli, R. M. Molecular imaging of proteins in tissues by mass spectrometry. Proc. Natl. Acad. Sci. 2008, 105 (47), 18126-18131.
(19) Su, P.; McGee, J. P.; Durbin, K. R.; Hollas, M. A. R.; Yang, M.; Neumann, E. K.; Allen, J. L.; Drown, B. S.; Butun, F. A.; Greer, J. B.; et al. Highly multiplexed, label-free proteoform imaging of tissues by individual ion mass spectrometry. Science Advances 2022, 8 (32), eabp9929. DOI: doi:10.1126/sciadv.abp9929.
(20) McGee, J. P.; Su, P.; Durbin, K. R.; Hollas, M. A. R.; Bateman, N. W.; Maxwell, G. L.; Conrads, T. P.; Fellers, R. T.; Melani, R. D.; Camarillo, J. M.; et al. Automated imaging and identification of proteoforms directly from ovarian cancer tissue. Nat. Commun. 2023, 14 (1), 6478. DOI: 10.1038/s41467-023-42208-3.
(21) Comi, T. J.; Neumann, E. K.; Do, T. D.; Sweedler, J. V. microMS: A Python Platform for Image-Guided Mass Spectrometry Profiling. J. Am. Soc. Mass Spectrom. 2017, 28 (9), 1919-1928. DOI: 10.1007/s13361-017-1704-1.
(22) Xie, Y. R.; Chari, V. K.; Castro, D. C.; Grant, R.; Rubakhin, S. S.; Sweedler, J. V. Data-Driven and Machine Learning-Based Framework for Image-Guided Single-Cell Mass Spectrometry. J. Proteome Res. 2023, 22(2), 491-500. DOI: 10.1021/acs.jproteome.2c00714.
(23) Shalek, A. K.; Satija, R.; Adiconis, X.; Gertner, R. S.; Gaublomme, J. T.; Raychowdhury, R.; Schwartz, S.; Yosef, N.; Malboeuf, C.; Lu, D.; et al. Single-cell transcriptomics reveals bimodality in expression and splicing in immune cells. Nature 2013, 498 (7453), 236-240. DOI: 10.1038/nature12172.
(24) Bankhead, P.; Loughrey, M. B.; Fernández, J. A.; Dombrowski, Y.; McArt, D. G.; Dunne, P. D.; McQuaid, S.; Gray, R. T.; Murray, L. J.; Coleman, H. G.; et al. QuPath: Open source software for digital pathology image analysis. Sci. Rep. 2017, 7 (1), 16878. DOI: 10.1038/s41598-017-17204-5.
(25) Kafader, J. O.; Melani, R. D.; Durbin, K. R.; Ikwuagwu, B.; Early, B. P.; Fellers, R. T.; Beu, S. C.; Zabrouskov, V.; Makarov, A. A.; Maze, J. T.; et al. Multiplexed mass spectrometry of individual ions improves measurement of proteoforms and their complexes. Nat. Methods 2020, 17 (4), 391-394. DOI: 10.1038/s41592-020-0764-5.
(26) Kafader, J. O.; Beu, S. C.; Early, B. P.; Melani, R. D.; Durbin, K. R.; Zabrouskov, V.; Makarov, A. A.; Maze, J. T.; Shinholt, D. L.; Yip, P. F. STORI plots enable accurate tracking of individual ion signals. Journal of The American Society for Mass Spectrometry 2019, 30 (11), 2200-2203.
(27) Weigand, M. R.; Yang, M.; Hu, H.; Zensho, C.; Laskin, J. Enhancement of lipid signals with ammonium fluoride in negative mode Nano-DESI mass spectrometry imaging. Int. J. Mass spectrom. 2022, 478, 116859. DOI: https://doi.org/10.1016/j.ijms.2022.116859.
(28) Horn, D. M.; Zubarev, R. A.; McLafferty, F. W. Automated reduction and interpretation of high resolution electrospray mass spectra of large molecules. J. Am. Soc. Mass Spectrom. 2000, 11 (4), 320-332. DOI: https://doi.org/10.1016/S1044-0305(99)00157-9.
(29) Mazorra-Carrillo, J. L.; De León-Rodríguez, A.; Huerta-Ocampo, J. A.; Velarde-Salcedo, A. J.; González de Mejia, E.; Barba de la Rosa, A. P. Proteomic analysis of chemically transformed NIH-3T3 cells reveals novel mechanisms of action of amaranth lunasin-like peptide. Food Res. Int. 2022, 157, 111374. DOI: https://doi.org/10.1016/j.foores.2022.111374.
(30) Tolmachev, A. V.; Udseth, H. R.; Smith, R. D. Radial stratification of ions as a function of mass to charge ratio in collisional cooling radio frequency multipoles used as ion guides or ion traps. Rapid Commun. Mass Spectrom. 2000, 14 (20), 1907-1913. DOI: https://doi.org/10.1002/1097-0231(20001030)14:20<1907::AID-RCM111>3.0.CO;2-M (accessed 2024/01/14).
(31) Kafader, J. O.; Melani, R. D.; Schachner, L. F.; Ives, A. N.; Patrie, S. M.; Kelleher, N. L.; Compton, P. D. Native vs Denatured: An in Depth Investigation of Charge State and Isotope Distributions. J. Am. Soc. Mass Spectrom. 2020, 31 (3), 574-581. DOI: 10.1021/jasms.9b00040.
(32) Derks, J.; Leduc, A.; Wallmann, G.; Huffman, R. G.; Willetts, M.; Khan, S.; Specht, H.; Ralser, M.; Demichev, V.; Slavov, N. Increasing the throughput of sensitive proteomics by plexDIA. Nat. Biotechnol. 2023, 41 (1), 50-59. DOI: 10.1038/s41587-022-01389-w.
(33) Webber, K. G. I.; Truong, T.; Johnston, S. M.; Zapata, S. E.; Liang, Y.; Davis, J. M.; Buttars, A. D.; Smith, F. B.; Jones, H. E.; Mahoney, A. C.; et al. Label-Free Profiling of up to 200 Single-Cell Proteomes per Day Using a Dual-Column Nanoflow Liquid Chromatography Platform. Anal. Chem. 2022, 94 (15), 6017-6025. DOI: 10.1021/acs.analchem.2c00646.

1. A method comprising:
moving a liquid droplet comprising a solvent across a substrate having disaggregated cells thereon;
sampling the moving liquid droplet with an ionization source, wherein sampling the moving liquid droplet comprises detecting a discrete cell extraction profile comprising a multiplicity of proteoform ions when the moving liquid droplet contacts a disaggregated cell on the substrate;
aggregating a mass-domain spectrum from a plurality of discrete cell extraction profiles;
assigning a single cell proteoform from the aggregated mass-domain spectrum; and
identifying one or more proteoforms from the assigned the single cell proteoform.

2. The method of claim 1, wherein the single cell proteoform is assigned by assigning a proteoform assignment score (PAS) as function of mass accuracy and relative abundance of isotopologue masses of ions.

3. The method of claim 2, wherein the proteoform assignment score is
$$PAS = 1 - {\prod }_{k = 1}^{n} [ 1 - ( {P}_{isotopologue} \times {P}_{mass error} ) ]$$
where P_{isotopologue} is the expected relative intensity of a matching isotopologue and P_{mass error} is a probability score for a mass error-based between the observed individual ion mass and a theoretical mass of the isotopologue.

4. The method of claim 3, wherein the proteoform assignment score utilizes a cumulative distribution function (CDF) of a normal distribution with a mean and standard deviation determined by the theoretical mass and width of an isotopic peak, respectively.

5. The method of claim 4, wherein P_{mass error} is
P_{mass error}∝{(CDF(mᵢₒₙ)mᵢₒₙ<mᵢₛₒ@[1−CDF(mᵢₒₙ)mᵢₒₙ>mᵢₛₒ))
where mᵢₒₙ is the mass of an individual ion and mᵢₛₒ is the theoretical mass of the matched isotopologue.

6. The method of claim 1, wherein the one or more proteoforms are identified by an intact mass tag search against candidate proteoforms and/or a deconvolution algorithm.

7. The method of claim 1, wherein the liquid droplet moves across the substrate at a rate that when the liquid droplet leaves the disaggregated cell the detection of the one or more proteoform ions wanes.

8. The method of claim 1, wherein the liquid droplet is rastered across the substrate.

9. (canceled)

10. The method of claim 1, wherein liquid droplet is moved across the substrate at a rate of 1 to 50 μm/s.

11. The method of claim 1, wherein a probe size of the liquid droplet is 25 to 400 μm.

12. The method of claim 1, wherein an injection time is about 200 to 900 msec.

13. The method of claim 1, wherein a higher-energy collisional dissociation gas pressure is 0.2 to 0.8

14. The method of claim 1, wherein the solvent comprises acetonitrile, water, and acetic acid.

15. The method of claim 14, wherein the solvent comprises between 1% and 5% acetic acid in 60%/40% acetonitrile/water.

16. The method of claim 1, wherein the flow rate of the solvent is between 150 and 500 nL/min.

17. The method of claim 1, wherein the substrate comprises a glass slide functionalized to facilitate cell adhesion.

18. The method of claim 1, wherein the cells are fixed onto the substrate by suspending cells in a buffer, fixing the suspended cells onto the substrate with a fixation solution comprising ethanol, and washing the disaggregated cells fixed on the substrate with a wash solution comprising ethanol and water.

19. (canceled)

20. The method of claim 18, further comprising removing the buffer prior to fixing the suspended cells onto the substrate with the fixation solution.

21. A system comprising:
a primary capillary tube configured to flow a solvent to a substrate having disaggregated cells thereon;
a nanospray capillary tube configured for analyte transfer, wherein the primary capillary tube and the nanospray capillary tube are configured to form a liquid droplet bridging a junction between the primary capillary tube and the nanospray capillary tube;
a movable stage, wherein the substrate having the disaggregated cells is disposed on the movable stage and the movable stage is configured to move the liquid droplet across the substrate;
an ionization source configured to sample the liquid droplet;
a detector configured to detect a discrete cell extraction profile comprising a multiplicity of proteoform ions; and
one or more processors configured to aggregate a mass-domain spectrum from a plurality of discreate cell extraction profiles, assign a single cell proteoform from the aggregated mass-domain spectrum, identify one or more proteoforms from the assigned the single cell proteoform, and/or control the movable stage to move the liquid droplet across the substrate.

22. The system of claim 21, wherein the one or more processors are configured to aggregate a mass-domain spectrum from a plurality of discreate cell extraction profiles, assign a single cell proteoform from the aggregated mass-domain spectrum, and identify one or more proteoforms from the assigned the single cell proteoform.
